(19) United States
(12) Patent Application Publication
(10) Pub. No.: US 2020/0256856 A1
(43) Pub. Date: Aug. 13, 2020

(54) SYSTEM AND METHODS OF IMAGE-BASED ASSAY USING CROF AND MACHINE LEARNING

(71) Applicant: Essenlix Corporation, Monmouth Junction, NJ (US)

(72) Inventors: Stephen CHOU, Princeton, NJ (US); Wei DING, East Windsor, NJ (US); Jun TIAN, Belle Mead, NJ (US); Wu CHOU, Basking Ridge, NJ (US); Yuecheng ZHANG, Yardley, PA (US); Mingquan WU, Princeton Junction, NJ (US); Li LI, Monmouth Junction, NJ (US); Michael LEONIY, Monmouth Junction, NJ (US)

(73) Assignee: Essenlix Corporation, Monmouth Junction, NJ (US)

(21) Appl. No.: 16/759,006

(22) PCT Filed: Oct. 26, 2018
(86) PCT No.: PCT/US18/57877

§ 371 Date: Apr. 24, 2020

Related U.S. Application Data
(60) Provisional application No. 62/577,481, filed on Oct. 26, 2017.

(51) Int. Cl. G01N 33/50; G01N 33/80; B01L 3/00; B01L 9/00; G01N 33/483; G01N 15/06; G06K 9/00; G06K 9/62

(52) U.S. Cl. CPC G01N 33/5094; G01N 2015/0073; B01L 3/508; B01L 9/523; G01N 33/4833; G01N 15/06; G06K 9/00134; G06K 9/6256; G06K 9/0014; B01L 2300/0609; B01L 2300/123; B01L 2300/168; B01L 2300/06; B01L 2300/025; B01L 2300/0816; G01N 33/80

(57) ABSTRACT

Among other things, the present invention is related to devices/apparatus and methods of performing cellular, biological, and chemical assays and procedures.

### CROSS REFERENCING

This application is a National Stage entry (§ 371) application of International Application No. PCT/US18/57877, filed on Oct. 26, 2018, which claims the benefit of U.S. Provisional Patent Application No. 62/577,481, filed on Oct. 26, 2017, the contents of which are relied upon and incorporated herein by reference in their entirety.

The entire disclosure of any publication or patent document mentioned herein is entirely incorporated by reference.

### FIELD

Among other things, the present invention is related to devices/apparatus and methods of performing cellular, biological, and chemical assays and procedures.

### BACKGROUND

In bio/chemical sensing and testing (e.g. immunoassay, nucleotide assay, blood cell counting, etc.), chemical reactions, and other processes, there are needs for methods and devices/apparatus that are fast, easy to conduct, inexpensive, and/or highly accurate. The present invention relates to the methods, devices, apparatus, and systems that address these needs.

### BRIEF DESCRIPTION OF THE DRAWINGS

The skilled artisan will understand that the drawings, described below, are for illustration purposes only. The drawings are not intended to limit the scope of the present teachings in any way. In some Figures, the drawings are in scale. In the figures that present experimental data points, the lines that connect the data points are for guiding a viewing of the data only and have no other means.

FIG. 1 provides a diagram that shows the structure of the QMAX device for image-based assay in the present invention

FIG. 2 provides a schematic diagram that shows an exemplary embodiment of using QMAX device for image-based assay in the present invention

FIG. 3 provides a schematic operation workflow diagram of an embodiment of the current invention, iMOST, based on QMAX device for assaying

FIG. 4 provides a schematic block diagram that shows the workflow for training machine learning model in the image-based assay

FIG. 5 provides a schematic block diagram that shows the workflow of predication/inference with the trained machine learning model in image-based assay

### DETAILED DESCRIPTION OF EXEMPLARY EMBODIMENTS

The following detailed description illustrates some embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It should be noted that the Figures do not intend to show the elements in strict proportion. For clarity purposes, some elements are enlarged when illustrated in the Figures. The dimensions of the elements in the Figure should be delineated from the descriptions herein provided and incorporated by reference.

### Definition

[1] Unless otherwise indicated, the methods, apparatus, operations, reagents, test procedures, and device features disclosed herein involve techniques, algorithms, and apparatus commonly used in microbiology, bio/chemical assay, microscopic imaging, image processing, optics, software, statistics, and computing, which are within the skill of the art. Such techniques and apparatus are known to those of skill in the art and are described in numerous texts and reference works.
[2] Unless defined otherwise, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice of the embodiments disclosed herein, some methods and materials are described.
[3] The headings provided herein are not intended to limit the disclosure.
[4] As used herein, the singular terms “a,” “an,” and “the” include the plural reference unless the context clearly indicates otherwise. The term “or” as used herein, refers to a non-exclusive or, unless otherwise indicated.
[5] The terms defined immediately below are more fully described by reference to the specification as a whole. It is to be understood that this disclosure is not limited to the particular methodology, protocols, and reagents described, as these may vary, depending upon the context they are used by those of skill in the art.
[6] The term “plurality” refers to more than one element. And the term “parameter value” herein refers to a numerical value that characterizes a physical property or a representation of that property. In some situations, a parameter value numerically characterizes a quantitative data set and/or a numerical relationship between quantitative data sets.
[7] The term “threshold” herein refers to any number that is used as, e.g. a cutoff to classify a sample feature as particular type of analyte, or a ratio of abnormal to normal cells in the sample. Threshold values can be identified empirically or analytically.
[8] The term “sample” refers to a specimen that is taken from and not limited to the substance in medical, biological, chemical, and physical process.
[9] The term “biological sample” refers to a sample, typically derived from a biological fluid, tissue, organ, etc. Such samples include, but are not limited to sputum/oral fluid, amniotic fluid, blood, urine, semen, stool, vaginal fluid, peritoneal fluid, pleural fluid, tissue explant, organ culture, cell culture, and any other tissue or cell preparation, or fraction or derivative thereof or isolated therefrom. The sample may be used directly as obtained from the biological source or following a pretreatment on the sample before being used in the assay. Methods of pretreatment may involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. In various embodiments, the biological sample is provided in a format that facilitates imaging for image-based assay. As an example, the biological sample may be stained and/or converted to a smear before being analyzed.
[10] The term “assay” refers to an investigative (analytic) procedure in and not limited to laboratory, medicine, pharmacology, environmental biology, healthcare, and molecular biology—for and not limited to qualitatively assessing or quantitatively measuring the presence, amount, concentration, or functional activity of a target entity (i.e. the analyte). The analyte can be a drug, a biochemical substance, or a cell in an organism or organic sample such as human blood.
[11] The term “image-based assay” refers to an assay procedure that utilizes the image of the sample taken by an imager, where the sample can be and not limited to medical, biological and chemical sample,
[12] The term “imager” refers to any device that can take image of the objects. It includes and not limited to cameras in the microscope, smartphone, or special device that can take image at various wavelength.
[13] The term “sample feature” refers to some property of the sample that represents a potentially interesting condition. In certain embodiments, a sample feature is a feature that appears in an image of a sample and can be segmented and classified by image processing including using machine learning with a machine learning model. Examples of sample features include and not limited to analyte types in the sample, e.g. red blood cells, white blood cells, and tumor cells, and it includes analyte count, shape, volume, concentration and the like.
[14] The term “machine learning” refers to algorithms, systems and apparatus in the field of artificial intelligence that often use statistical techniques and artificial neural network to give computer the ability to “learn” (i.e., progressively improve performance on a specific task) from data without being explicitly programmed.
[15] The term “artificial neural network” refers to a layered connectionist system inspired by the biological networks that can “learn” to perform tasks by considering examples, generally without being programmed with any task-specific rules.
[16] The term “convolution” refers to a particular mathematical operation on two functions (f and g) to produce a third function that expresses how the shape of one is modified by the other.
[17] The term “convolutional neural network” refers to a class of multilayer feed-forward artificial neural networks, most commonly applied to analyzing visual images utilizing convolution in its operations.
[18] The term “deep learning” refers to a broad class of machine learning methods in artificial intelligence (AI) that learn from data with some deep network structures.
[19] The term “machine learning model” refers to a trained computational model that is built from a training process in the machine learning from the data. The trained machine learning model is applied during the predication (inference) stage by the computer that gives computer the capability to perform certain tasks (e.g. detect and classify the objects) on its own. Examples of machine learning models include ResNet, DenseNet, etc. which are also named as “deep learning models” because of the layered depth in their network structure.
[20] The term “image segmentation” refers to an image analysis process that partitions a digital image into multiple segments (sets of pixels, often with a set of bit-map masks that cover the image segments enclosed by their segment boundary contours). Image segmentation can be achieved through the image segmentation algorithms in image processing, such as watershed, Otsu method, grabcuts, mean-shift, etc., and also through machine learning algorithms, such as MaskRCNN, etc.
[21] The term “pseudo-2D image” refers to an image not actually but having the appearance of a 2D image. For instance, in the context of the present invention, the image taken by the imager on the sample holding device is typically a pseudo-2D image, because it has the appearance of a 2D image but it is an image of a 3-D sample with its depth being known or characterized through other means.
[22] The term “heatmap” refers to a two-dimensional representation of information according to certain metrics often assisted with colors for its graphic visualization.
[23] The term “signal list processing” refers to processing the information from a list of items. For instance, signals from potential analytes can be put in a list data structure, and in the signal list processing, each item in the list is processed to determine its identity.
[24] The term “local searching process” refers to a search process which is limited to a local region, and the term “local signal peaks” refers to the signal peaks found in the local searching process.
[25] The term “true analyte” refers to the detected analyte being a true one, not from a false detection, and the term “false analyte” refers to the detected analyte not a true one but from a false detection.
[26] The term “blob detection” refers to a class of methods aimed at detecting regions in a digital image that differ in properties, such as brightness or color, compared to surrounding regions. Informally, a blob is a region of an image in which some properties are constant or approximately constant.
[27] The term “adaptative thresholding” refers to the thresholding methods whose value at each pixel location depends on the neighboring pixel intensities. In image processing, adaptative thresholding typically takes a grayscale or color image as input and, in the simplest implementation, outputs a binary image representing the segmentation, wherein for each pixel in the image, a threshold has to be calculated. Examples of adaptative thresholding in image processing includes Otsu's method which performs clustering-based image thresholding in image segmentation.
[28] The term “detection model by convolution” refers to a detection model that utilizes the convolution operations to detect and classify the input signal.

### Assay and Imaging Using CROF and Machine Learning

### 1. QMAX Device for Assay and Imaging

Among other things, the present invention relates to using artificial intelligence to analyze samples.

In some embodiments, the sample is held in a sample holder such as but not limited to a QMAX device that is disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, an imager is used to capture one or more images of a biological sample in the sample holder, wherein the analyte count, concentration and location of analytes contained in the sample can be obtained. In some embodiments, the images are submitted to a computing unit. The computing unit can physically be connected to the imager, connected through network, or in-directly through image transfer.

One type of biological sample considered herein is human blood and its components include the Red Blood Cells/Corpuscles (RBC) also called “Erythrocytes”, White Blood Cells/Corpuscles (WBC) also called “Leukocytes”, and Platelets (PLT), where hemoglobin is the protein molecule in RBC that carries oxygen from the lungs to the body tissues and returns carbon dioxide from the tissues back to the lungs. These vital components in the blood are minute objects. For instance, the largest WBC has a ball shape of only 12˜15 μm in diameter, RBC has a disk shape with a height of ˜2 μm and a diameter around 7.5 μm, and PLT is even much smaller with a diameter only around 1˜2 μm, with a size less than 20% of RBC.

Blood tests, especially the complete blood count (CBC), are most widely administered because they are key health indicators for humans. For example, the concentration of WBC in the blood is a strong indicator of infection, abnormality of immune system, effects or side-effects of drugs and medical treatments, etc. Moreover, results from CBC tests are often used as an indicator to screen patients for many life-threatening sickness, such as leukemia, in which an early indication from blood test, such as CBC, can result in the saving of lives.

In addition to their microscopic sizes, various types of blood cells have huge differences in concentration, and they vary with a large range, making accurate blood test challenging. Conventional blood testing, especially the CBC, is conducted in the professional test labs using sophisticated machineries operated by trained experts. The present invention with QMAX device is to provide high accuracy test readings of CBC using commodity devices such as smartphones.

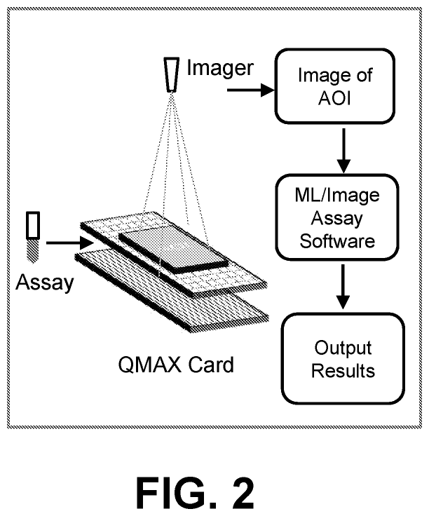

FIG. 2 provides a schematic diagram that shows an exemplary embodiment based on QMAX device for image-based assay in the present invention. The blood sample for assaying is loaded into the QMAX device as illustrated in the diagram of FIG. 2. The QMAX device in the embodiment of the present invention has two parallel plates and a gap that is made intentionally narrow, proportional to the size of the analyte for assaying. As such, analytes sandwiched between the said plates for assaying form a single-layer and can be imaged from the top plate over the area-of-interest (colored in yellow color) by an imager. The image of the analytes taken by the imager over the area-of-interest (AoI) is fed to the predication/inference module of the system pre-loaded with a machine learning model for assaying the analytes in image-based assay.

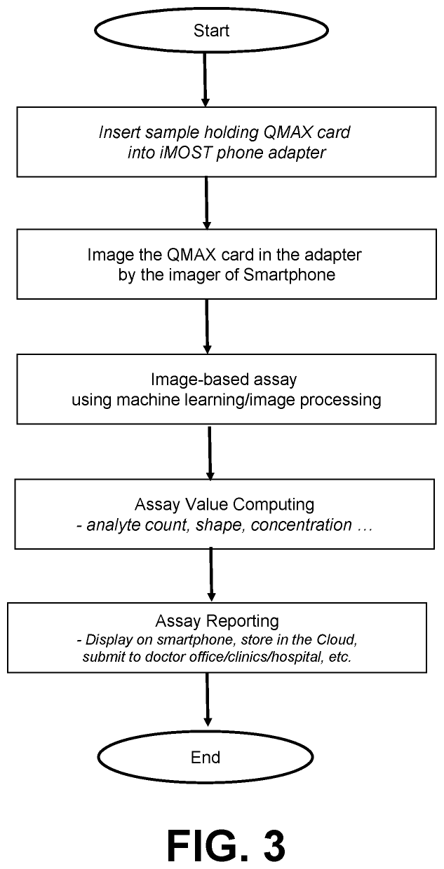

FIG. 3 depicts a schematic diagram of an embodiment of the current invention, iMOST, that is based on a smartphone (e.g. iPhone 6) and FIG. 6 is an image of iMOST system for assaying based on QMAX device, wherein a specially designed phone adapter is mounted on the smartphone and it uses the camera of the smartphone as the imager for image-based assay. In operation of assaying, the sample holding device, QMAX device, is inserted into the phone adapter and the image of the QMAX device is taken by an QMAX imager—the camera of the smartphone in iMOST, over the AoI on the upper plate of the QMAX device. The image of the QMAX device is fed to the predication/inference module of iMOST as input, and it is processed by the image-based assay module of iMOST with a pre-loaded machine learning model to detect the analytes in the image of the sample. The information obtained in the predication/interference module of iMOST is fed to its analysis module to perform assay value computation to determine the analytes properties in the sample for assaying—including the total assay count, shape, concentration, etc. The detected values and properties from iMOST assay value computation module can be displayed directly on the smartphone, uploaded and archived in the iMOST Cloud, or submitted to doctor's office/clinics/hospitals for recording and follow-up actions.

QMAX device is useful to provide accurate assaying using commodity devices such as smartphones. It can be performed in public without requiring a special test lab environment. As such, images of the sample taken by the imager on QMAX devices for assaying can have a huge range of variations and a much higher level of noises—a situation not seen in professional test machines of the prior arts. As a consequence, traditional approaches for assaying in CBC will not be able to achieve the desired high precision in non-lab environment with commodity devices (e.g. cameras from smartphones). A key idea in the present invention is to innovatively formulate and model the assaying for analyte detection and concentration measurement in a machine learning framework that is in combination of the use of a QMAX type device—upon which machine learning methods/algorithms can be applied to discriminatively detect, locate, count, and obtain the concentration of various types of analytes in the sample for assaying—including the blood cells in the sample for CBC. Moreover, it can achieve accurate assaying in the presence of imperfections—including working in non-lab environment and using non-specialized consumer hardware, e.g. smartphones.

Blood cell distribution and concentration measurement in a machine learning framework—upon which machine learning methods/algorithms can be applied to discriminatively detect, locate, count, and obtain the concentration of all types of blood cells in the assay. Moreover, it can achieve high accuracy in the presence of imperfections and variations from using non-specialized consumer devices.

However, CBC is extremely challenging, because it needs to quantify the blood cell (analyte) concentration, and this is beyond the detection of certain cells in the blood. To obtain the high accuracy in CBC, it needs to precisely characterize both the volume and the count of the blood cells (analytes) in the sample for assaying, because the concentration C is the ratio of these two quantities:

$$C = \frac{Count}{Volume}$$

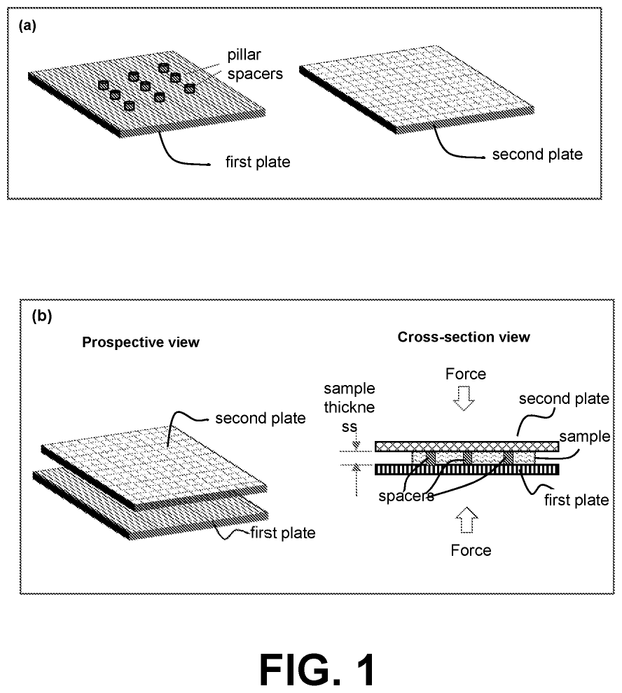

This issue becomes more acute in the iMOST for assaying in non-lab environment such as point-of-service and mobile health. Among other things, the present invention relates to using artificial intelligence to analyze samples, such as blood samples, and it makes an innovative use of the sample holding QMAX device to overcome the limitations in prior arts. In one aspect, the present invention provides a machine learning framework to improve the functionality of using QMAX device in assaying when a computer program is used. The novel approach of using QMAX type device in combination with a machine learning framework is described as follows:
a) The sample for assaying is loaded into the sample holding QMAX device and is kept between two parallel plates separated by a narrow gap with the upper plate being transparent for imaging. FIG. 1 illustrates the construction of the QMAX device in detail. Small pillars are fabricated on the base plate and they are distributed in a special pattern to make the gap between plates uniform. The gap between the plates is spaced narrowly—with the distance of the gap being proportional to the size of the analytes to be assayed—by which the analytes in the sample form a single layer between the said plates. As such, the sample volume corresponding to the AoI (area-of-interest) on the upper plate can be precisely characterized by: Volume=AoI×gap because of the uniformity of the gap between the plates.
b) Obtain pseudo-2D image of the sample in AoI×gap: In QMAX, the gap distance between the two plates is made intentionally narrow, uniform and known priori—proportional to the size of the analytes, such that analytes in the sample form a single layer on the base plate of the QMAX device. As such, these analytes can be captured in the image of the AoI taken by the QMAX imager on sample holding QMAX device. Moreover, the image taken by the said imager on the sample holding QMAX device is a special pseudo-2D image, because it has the appearance of a 2D image but it is an image of a 3-D sample with its depth being known or characterized through other means.
c) The captured pseudo-2D image taken over the AoI of the QMAX device can characterize both the amount of analytes and the volume of the sample under AoI for assaying through a) and b), upon which the analyte concentration in the sample can be determined.
d) Based on a), b) and c), the CBC and assaying in iMOST become amendable in a machine learning framework based on the captured image over the selected AoI taken by the QMAX imager on the sample holding QMAX device—because the associated analyte counts and sample volume can be characterized precisely from the captured pseudo-2D image and the gap between the plates.
e) Based on a), b), c) and d), the framework described herein applies to analyte detection, localization, identification, segmentation and counting in CBC and other tests alike.
f) Based on a), b), c) and d), the framework described herein apply to intelligently selecting AoI in assaying to improve the accuracy and reliability of CBC and other tests.

However, images from the QMAX imager are often noisy, especially when the commodity devices are used. The captured image contains not only the pseudo-2D image of analytes, but also the noise and artifacts—including and not limited to air bobbles, dusts, shadows, and pillars. To obtain high accuracy results for CBC and other tests, it needs to precisely count the analytes in the sample and estimate the volume associated with the analytes in assaying. In certain embodiments, the present invention provides a machine learning framework of QMAX based devices that allows to apply algorithms such as deep learning to discriminatively locate, identify, segment and count analytes (e.g. blood cells) based on the said pseudo-2D image captured by the QMAX imager.

### 2. Workflow

One aspect of the present invention provides a framework of machine learning and deep learning for analyte detection and localization. A machine learning algorithm is an algorithm that is able to learn from data. A more rigorous definition of machine learning is “A computer program is said to learn from experience E with respect to some class of tasks T and performance measure P, if its performance at tasks in T, as measured by P, improves with experience E.” It explores the study and construction of algorithms that can learn from and make predictions on data—such algorithms overcome the static program instructions by making data driven predictions or decisions, through building a model from sample inputs.

Deep learning is a specific kind of machine learning based on a set of algorithms that attempt to model high level abstractions in data. In a simple case, there might be two sets of neurons: ones that receive an input signal and ones that send an output signal. When the input layer receives an input, it passes on a modified version of the input to the next layer. In a deep network, there are many layers between the input and output (and the layers are not made of neurons but it can help to think of it that way), allowing the algorithm to use multiple processing layers, composed of multiple linear and non-linear transformations.

One aspect of the present invention is to provide two analyte detection and localization approaches. The first approach is a deep learning approach and the second approach is a combination of deep learning and computer vision approaches.

### 1^{st} Approach—Deep Learning Approach

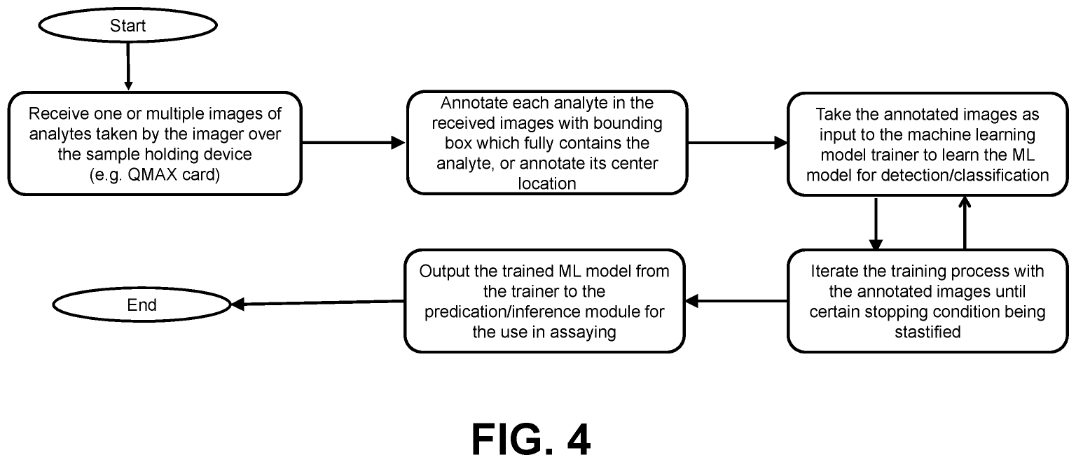

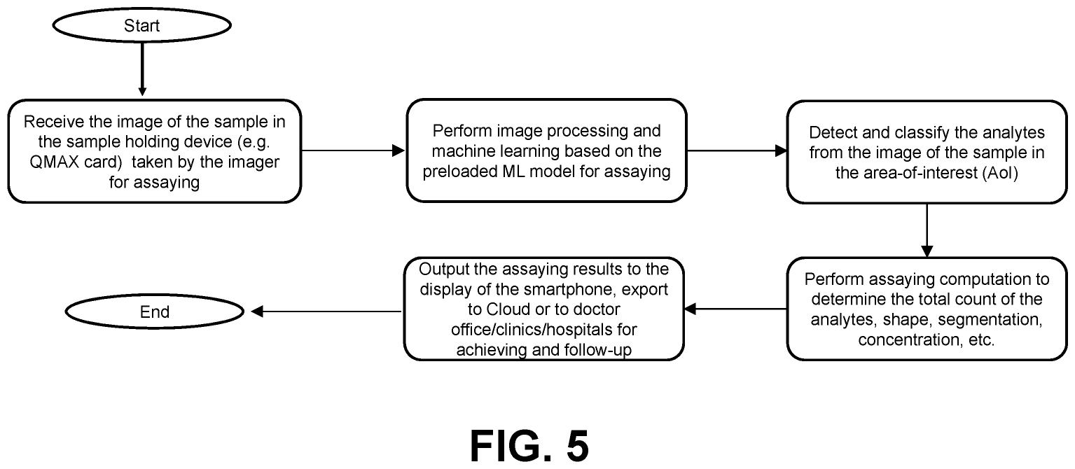

In the first approach, the disclosed analyte detection and localization workflow consists of two stages, training and prediction. FIG. 4 and FIG. 5 depict the both the training and the predication stage. We describe training and prediction stages in the following paragraphs.

### (i) Training Stage

In the training stage, training data with annotation is fed into a convolutional neural network. Convolutional neural network is a specialized neural network for processing data that has a grid-like, feed forward and layered network topology. Examples of the data include time-series data, which can be thought of as a 1D grid taking samples at regular time intervals, and image data, which can be thought of as a 2D grid of pixels. Convolutional networks have been successful in practical applications. The name “convolutional neural network” indicates that the network employs a mathematical operation called convolution. Convolution is a specialized kind of linear operation. Convolutional networks are simply neural networks that use convolution in place of general matrix multiplication in at least one of their layers.

FIG. 4 is a schematic block diagram that shows the workflow for training machine learning model in the image-based assay. It receives one or multiple images of samples that contain the analytes taken by the imager over the sample holding QMAX device as training data. Training data are annotated for analytes to be assayed, wherein the annotations indicate whether or not analytes are in the training data and where they locate in the image. Annotation can be done in the form of tight bounding boxes which fully contains the analyte, or center locations of analytes. In the latter case, center locations are further converted into circles covering analytes or a Gaussian kernel in a point map.

When the size of training data is large, training machine learning model presents two challenges: annotation (usually done by human) is time consuming, and the training is computationally expensive. To overcome these challenges, one can partition the training data into patches of small size, then annotate and train on these patches, or a portion of these patches. The term “machine learning” refers to algorithms, systems and apparatus in the field of artificial intelligence that often use statistical techniques and artificial neural network trained from data without being explicitly programmed.

As illustrated in FIG. 4, the annotated images are fed to the machine learning (ML) training module, and the model trainer in the machine learning module will train a ML model from the training data (annotated sample images). The input data will be fed to the model trainer in multiple iterations until certain stopping criterion is satisfied. The output of the ML training module is a ML model—a computational model that is built from a training process in the machine learning from the data that gives computer the capability to perform certain tasks (e.g. detect and classify the objects) on its own.

The trained machine learning model is applied during the predication (or inference) stage by the computer. Examples of machine learning models include ResNet, DenseNet, etc. which are also named as “deep learning models” because of the depth of the connected layers in their network structure. In some embodiments, the Caffe library with fully convolutional network (FCN) was used for model training and predication, and other convolutional neural network architecture and library can also be used, such as TensorFlow.

The training stage generates a model that will be used in the prediction stage. The model can be repeatedly used in the prediction stage for assaying the input. Thus, the computing unit only needs access to the generated model. It does not need access to the training data, nor requiring the training stage to be run again on the computing unit.

### (ii) Prediction Stage

As illustrated in FIG. 5, in the predication/inference stage, a detection component is applied to the input image, and an input image is fed into the predication (inference) module preloaded with a trained model generated from the training stage. The output of the prediction stage can be bounding boxes that contain the detected analytes with their center locations or a point map indicating the location of each analyte, or a heatmap that contains the information of the detected analytes.

When the output of the prediction stage is a list of bounding boxes, the number of analytes in the image of the sample for assaying is characterized by the number of detected bounding boxes. When the output of the prediction stage is a point map, the number of analytes in the image of the sample for assaying is characterized by the integration of the point map. When the output of the prediction is a heatmap, a localization component is used to identify the location and the number of detected analytes is characterized by the entries of the heatmap.

One embodiment of the localization algorithm is to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list. In the detection component using heatmap, an input image, along with the model generated from the training stage, is fed into a convolutional neural network, and the output of the detection stage is a pixel-level prediction, in the form of a heatmap. The heatmap can have the same size as the input image, or it can be a scaled down version of the input image, and it is the input to the localization component. We disclose an algorithm to localize the analyte center. The main idea is to iteratively detect local peaks from the heatmap. After the peak is localized, we calculate the local area surrounding the peak but with smaller value. We remove this region from the heatmap and find the next peak from the remaining pixels. The process is repeated only all pixels are removed from the heatmap.

In certain embodiments, the present invention provides the localization algorithm to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list.

Algorithm GlobalSearch (heatmap)
Input:

heatmap

Output:

loci

loci ← [symbol]
sort(heatmap)
while (heatmap is not empty) {

s ← pop(heatmap)
D ← {disk center as s with radius R}
heatmap = heatmap \ D // remove D from the heatmap
add s to loci

}

After sorting, heatmap is a one-dimensional ordered list, where the heatmap value is ordered from the highest to the lowest. Each heatmap value is associated with its corresponding pixel coordinates. The first item in the heatmap is the one with the highest value, which is the output of the pop(heatmap) function. One disk is created, where the center is the pixel coordinate of the one with highest heatmap value. Then all heatmap values whose pixel coordinates resides inside the disk is removed from the heatmap. The algorithm repeatedly pops up the highest value in the current heatmap, removes the disk around it, till the items are removed from the heatmap.

In the ordered list heatmap, each item has the knowledge of the proceeding item, and the following item. When removing an item from the ordered list, we make the following changes, as illustrated in FIG. 2:
Assume the removing item is xᵣ, its proceeding item is xₚ, and its following item is x_{f}.
For the proceeding item xₚ, re-define its following item to the following item of the removing item. Thus, the following item of xₚ is now x_{f}.
For the removing item x₁, un-define its proceeding item and following item, which removes it from the ordered list.
For the following item x_{f}, re-define its proceeding item to the proceeding item of the removed item. Thus, the proceeding item of x_{f} is now xₚ.

After all items are removed from the ordered list, the localization algorithm is complete. The number of elements in the set loci will be the count of analytes, and location information is the pixel coordinate for each s in the set loci.

Another embodiment searches local peak, which is not necessary the one with the highest heatmap value. To detect each local peak, we start from a random starting point, and search for the local maximal value. After we find the peak, we calculate the local area surrounding the peak but with smaller value. We remove this region from the heatmap and find the next peak from the remaining pixels. The process is repeated only all pixels are removed from the heatmap.

Algorithm LocalSearch (s, heatmap)
Input:

s: starting location (x, y)
heatmap

Output:

s: location of local peak.

We only consider pixels of value > 0.
Algorithm Cover (s, heatmap)
Input:

s: location of local peak.
heatmap:

Output:

cover: a set of pixels covered by peak:

This is a breadth-first-search algorithm starting from s, with one altered condition of visiting points: a neighbor p of the current location q is only added to cover if heatmap[p]>0 and heatmap[p]<=heatmap[q]. Therefore, each pixel in cover has a non-descending path leading to the local peak s.

Algorithm Localization (heatmap)
Input:

heatmap

Output:

loci

loci ←{ }
pixels ←{all pixels from heatmap}
while pixels is not empty {

| s ←any pixel from pixels | |
|---|---|
| s ←LocalSearch(s, heatmap) | // s is now local peak |

| probe local region of radius R surrounding s for better local peak | |
|---|---|
| r ←Cover(s, heatmap) | |
| pixels ← pixels \ r | // remove all pixels in cover |

add s to loci

### Mixture of Deep Learning and Computer Vision Approaches

In the second approach, the detection and localization are realized by computer vision algorithms, and a classification is realized by deep learning algorithms, wherein the computer vision algorithms detect and locate possible candidates of analytes, and the deep learning algorithm classifies each possible candidate as a true analyte and false analyte. The location of all true analyte (along with the total count of true analytes) will be recorded as the output.

### Detection

The computer vision algorithm detects possible candidate based on the characteristics of analytes, including but not limited to intensity, color, size, shape, distribution, etc.

A pre-processing scheme can improve the detection. Pre-processing schemes include contrast enhancement, histogram adjustment, color enhancement, de-nosing, smoothing, de-focus, etc.

After pre-processing, the input image is sent to a detector. The detector tells the existing of possible candidate of analyte and gives an estimate of its location. The detection can be based on the analyte structure (such as edge detection, line detection, circle detection, etc.), the connectivity (such as blob detection, connect components, contour detection, etc.), intensity, color, shape using schemes such as adaptive thresholding, etc.

### Localization

After detection, the computer vision algorithm locates each possible candidate of analytes by providing its boundary or a tight bounding box containing it. This can be achieved through object segmentation algorithms, such as adaptive thresholding, background subtraction, floodfill, mean shift, watershed, etc. Very often, the localization can be combined with detection to produce the detection results along with the location of each possible candidates of analytes.

### Classification

The deep learning algorithms, such as convolutional neural networks, achieve start-of-the-art visual classification. We employ deep learning algorithms for classification on each possible candidate of analytes. Various convolutional neural network can be utilized for analyte classification, such as VGGNet, ResNet, MobileNet, DenseNet, etc.

Given each possible candidate of analyte, the deep learning algorithm computes through layers of neurons via convolution filters and non-linear filters to extract high-level features that differentiate analyte against non-analytes. A layer of fully convolutional network will combine high-level features into classification results, which tells whether it is a true analyte or not, or the probability of being a analyte.

### 3. Example of Present Invention

A1. A method and apparatus for complete blood count (CBC) in diagnostics from the blood sample based on one or more devices and algorithms, comprising:
(a) receiving the blood sample for assaying and loading the sample into a sample holding device such as a QMAX device;
(b) the said sample holding device having two parallel placed plates—with the gap in between being made uniform—controlled precisely by the properly distributed pillars between them wherein the volume of the sample for assaying under the area-of-interest (AoI) at the top plate can be characterized by the area of AoI and the said gap;
(c) the said gap between the plates being intentionally spaced narrowly—with the distance of the gap being proportional to the size of the blood cells (analytes)—by which the blood cells (analytes) in the assay form a single layer between the plates;
(d) the said upper plate is transparent to an imager such as a QMAX imager, so that the image of the blood cells (analytes) in the AoI and between the said plates can be captured in the image taken by the said imager from the upper plate;
(e) the said blood cells (analytes) captured in the image over AoI by the said imager are pseudo-2D objects, whose volume in the sample can be characterized based on their area in the image of the selected AoI and the said gap;
(f) generating the list of detected pseudo-2D objects from the image taken by the said imager over the AoI, using algorithms of machine learning and image processing;
(g) calculating the concentration and segmentation of the detected analytes from
   i. the list of detected pseudo-2D objects in the image taken by the imager over the selected AoI, and
   ii. the relation of the analytes in the said image and their associated volume in the sample for assaying based on (b) and (c); and
(h) storing the calculated location, count, concentration, etc. of the detected blood cells (analytes) in the storage device or displaying the assaying results on the screen of a computer or a mobile device.
   A2. The method and apparatus of embodiment A1 wherein the machine learning comprises:
a. collecting images taken by the said imager over multiple AoIs from the images taken over the sample holding QMAX device, and labeling the analyte (i.e. blood cell) in the image to generate the annotated data set, wherein each analyte is represented by a tight bounding box surrounding it or a point map from a local intensity heatmap or a local distribution (e.g. Gaussian) kernel;
b. feeding the annotated data set to a convolutional neural network for model training, wherein the output of the model training is a detection model to detect and identify the analytes in the assaying from the image taken by the said imager; and
c. in assaying, feeding the image of AoI taken by the said imager to the said detection model, generating
   i. the list of detected analytes with their positions in the AoI,
   ii. calculating the total count of the analytes being captured, classifying them into various classes, determining their shape and concentration in the sample for assaying.
      A3. The method and apparatus of embodiment A1, wherein the system comprises of a device, an imager and computing units:
(a) the device is configured to compress at least part of a test sample into a layer of highly uniform thickness;
(b) the imager is configured to produce an image of the sample at the layer of uniform thickness, wherein the image includes detectable signals from analytes in the test sample;
(c) the computing unit is configured to:
   i. receive the image from the imager;
   ii. analyze the image with a detection model and generate a 2-D data array of the image, wherein the 2-D data array includes the probability or likelihood data of the analyte for being at each location in the image, and the detection model is established through a training process that comprises:
      feeding an annotated data set to a convolutional neural network, wherein the annotated data set is from samples that are the same type as the test sample and for the same analyte; and
      training and establishing the detection model by convolution; and
   iii. in testing, feeding the data to the model, generating and analyzing the 2-D data array to detect local signal peaks with signal list processing, or local search processing to detect the analyte; and
   iv. calculate the amount of the analyte being detected based on local signal peak information and the analyte relation to the assay volume.
      A4. The method and apparatus of embodiment A3, wherein the imager comprises a camera.
      A5. The method and apparatus of embodiment A3, wherein the camera is part of a mobile communication device such as a smart phone.
      A6. The method and apparatus of embodiment A1 or A3, wherein the computing unit is part of a mobile communication device.
      A7. The method and apparatus of embodiment A1 or A3, wherein a method of mixture of computer vision and deep learning for data analysis is used, comprising:
(a) receiving an image of a test sample, wherein the sample is loaded into a QMAX device and the image is taken by an imager connected to the QMAX device, wherein the image includes detectable signals from an analyte in the test sample;
(b) analyzing the image with a detection algorithm that finds possible candidate based on the characteristics of analytes;
(c) analyzing the image with a localization algorithm that locates each possible candidate of analytes by providing its boundary or a tight bounding box containing it;
(d) analyzing the image with a deep learning algorithm that classifies each possible candidate as a true analyte and false analyte;
(e) outputting the locations of true analytes, the total count of true analytes and the concentration of the analytes in the assay.
   A8. The method and apparatus of embodiment A1, A3 or A7, where the detection is based on the analyte structure (such as edge detection, line detection, circle detection, etc.).
   A9. The method and apparatus of embodiment A1, A3 or A7, where the detection is based on the connectivity (such as blob detection, connect components, contour detection, etc.).
   A10. The method and apparatus of embodiment A1, A3 or A7, where the detection is based on intensity, color, shape using schemes such as adaptive thresholding, etc.
   A11. The method and apparatus of embodiment A1, A3 or A7, where the detection is enhanced by a pre-processing scheme.
   A12. The method and apparatus of embodiment A1, A3 or A7, where the localization is based on object segmentation algorithms, such as adaptive thresholding, background subtraction, flood fill, mean shift, watershed, etc.
   A13. The method and apparatus of embodiment A1, A3 or A7, where the localization is combined with detection to produce the detection results along with the location of each possible candidates of analytes.
   A14. The method and apparatus of embodiment A1, A3 or A7, where the detection and classification are based on machine learning, such as convolutional neural networks.
   A15. The method and apparatus of embodiment A1, A3 or A7, wherein the assay is held between two evenly spaced plates with an intentionally narrowed gap which is in proportion to the diameter of the analyte.
   A16. The method and apparatus of embodiment A1, A3 or A7, wherein one plate of the said device is transparent, so that the AoI (area-of-interest) on the said plate can be imaged to reveal the pseudo-2D layer of the analytes sandwiched between the two narrowly spaced plates.
   A17. The method and apparatus of embodiment A1, A3 or A7, whereas the embodiment is diagnostic, chemical or biological test generally.
   AA1. A method and apparatus for complete blood count (CBC) in diagnostics from the blood assay based on one or more devices and algorithms, comprising:
(i) receiving the blood assay and loaded into a QMAX device;
(j) the said device comprises of two parallel placed plates—with the gap in between being uniform—controlled precisely by the properly distributed pillars between them, whereas the volume of the assay under the area-of-interest (AoI) at the top plate can be characterized by the area of AoI and the gap;
(k) whereas the said plates are intentionally spaced narrowly—with a gap proportional to the size of the blood cells—by which the blood cells in the assay form a single layer between the plates;
(l) whereas the said upper plate is transparent to QMAX imager, so that the image of the blood cells in the AoI and between the plates can be captured by QMAX imager;
(m) whereas the said blood cells captured in the image over AoI by the imager are pseudo-2D objects and they relate to the volume in the assay characterized by the selected AoI and the said gap;
(n) generating the list of detected pseudo-2D objects from the image taken by the QMAX imager over the AoI, using algorithms of machine learning and image processing;
(o) calculating the concentration of the detected analytes from
   iii. the list of detected pseudo-2D objects in the image taken by the imager over the selected AoI, and
   iv. the relation of the analytes with the volume of the assay based on (b) and (c); and
(p) storing the location, count, and concentration of the detected blood cells (analytes) in the storage device or displaying the test results on the screen of a computer or a mobile device.
   AA2. The method and apparatus of embodiment A1 wherein the machine learning comprises:
d. collecting images taken by the said imager over multiple AoIs and labeling the blood cell signals in the image to generate the annotated data set;
e. feeding the annotated data set to a convolutional neural network, wherein the training and establishing the detection model by convolution; and
f. in CBC test, feeding the AoI image data to the said detection model, analyzing the 2-D data array to detect local signal peaks with:
   iii. signal list processing, or
   iv. local searching processing; and calculating the amount of analytes being captured based on local signal peak information and the assay volume associated with the AoI in the assay.
      AA3. The method and apparatus of embodiment A2, wherein the signal list processing comprises:
a. establishing a signal list by iteratively detecting local peaks from the 2-D data array, calculating a local area surrounding the detected local peak, and removing the detected peak and the local area data into the signal list in rank order; and
b. sequentially and repetitively removing highest signals from the signal list and signals from around the highest signal, thus detecting local signal peaks.
   AA4. The method and apparatus of embodiment A2, wherein the local search process comprises:
a. searching for a local maximal value in the 2-D data array by starting from a random point;
b. calculating the local area surrounding the peak but with smaller value;
c. removing the local maximal value and the surrounding smaller values from the 2-D data array; and
d. repeating steps a-c to detect local signal peaks.
   B1. A machine learning framework at microscopic cell distribution level to detect, locate, count and obtain all types of analyte concentrations with method of deep learning for data analysis, comprising:
(a) receiving an image of a test sample, wherein the sample is loaded into a QMAX device and the image is taken by an imager connected to the QMAX device, wherein the image includes detectable signals from an analyte in the test sample;
(b) analyzing the image with a detection model and generating a 2-D data array of the image, wherein the 2-D data array includes probability data of the analyte for each location in the image, and the detection model is established through a training process that comprises:
   i. feeding an annotated data set to a convolutional neural network, wherein the annotated data set is from samples that are the same type as the test sample and containing the same type of analytes for assaying; and
   ii. training and establishing the detection model with convolution; and analyzing the 2-D data array to detect local signal peaks with:
   v. signal list process, or
   vi. local searching process; and
(c) calculating the amount of the analytes based on local signal peak information.
   B2. The method of embodiment B1, wherein the signal list process comprises:

(a) establishing a signal list by iteratively detecting local peaks from the 2-D data array, calculating a local area surrounding the detected local peak, and removing the detected peak and the local area data into the signal list in order; and

(b) sequentially and repetitively removing highest signals from the signal list and signals from around the highest signal, thus detecting local signal peaks.

B3. The method of any B embodiments, wherein the local search process comprises:

(a) searching for a local maximal value in the 2-D data array by starting from a random point;

(b) calculating the local area surrounding the peak but with smaller value;

(c) removing the local maximal value and the surrounding smaller values from the 2-D data array; and

(d) repeating steps i-iii to detect local signal peaks.

B4. The method of any prior method embodiments, wherein the annotated data set is partitioned before annotation.
B5. A system for data analysis, comprising:

a QMAX device, an imager, and computing unit, wherein:

(a) the QMAX device is configured to compress at least part of a test sample into a layer of highly uniform thickness;

(b) the imager is configured to produce an image of the sample at the layer of uniform thickness, wherein the image includes detectable signals from an analyte in the test sample;

(c) the computing unit is configured to:
i. receive the image from the imager;
ii. analyze the image with a detection model and generate a 2-D data array of the image, wherein the 2-D data array includes probability data of the analyte for each location in the image, and the detection model is established through a training process that comprises: feeding an annotated data set to a convolutional neural network, wherein the annotated data set is from samples that are the same type as the test sample and contain the same type of analytes for assaying; training and establishing the detection model with convolution; and
iii. analyzing the 2-D data array to detect local signal peaks with signal list process, or local searching process; and
iv. calculate the amount of the analytes based on local signal peak information.
   B6. The system of embodiment B5, wherein the imager comprises a camera.
   B7. The system of embodiment B6, wherein the camera is part of a mobile communication device.
   B8. The system of any prior B embodiments, wherein the computing unit is part of a mobile communication device.
   C1. A method of mixture of computer vision and deep learning for data analysis, comprising:
(f) receiving an image of a test sample, wherein the sample is loaded into a QMAX device and the image is taken by an imager connected to the QMAX device, wherein the image includes detectable signals from an analyte in the test sample;
(g) analyzing the image with a detection algorithm that finds possible candidate based on the characteristics of analytes;
(h) analyzing the image with a localization algorithm that locates each possible candidate of analytes by providing its boundary or a tight bounding box containing it;
(i) analyzing the image with a deep learning algorithm that classifies each possible candidate as a true analyte and false analyte;
(j) outputting the locations of true analytes and the total count of true analytes.
   C2. The system of embodiment C1, where the detection is based on the analyte structure (such as edge detection, line detection, circle detection, etc.).
   C3. The system of embodiment C1, where the detection is based on the connectivity (such as blob detection, connect components, contour detection, etc.).
   C4. The system of embodiment C1, where the detection is based on intensity, color, shape using schemes such as adaptive thresholding, etc.
   C5. The system of embodiment C1, where the detection is enhanced by a pre-processing scheme.
   C6. The system of embodiment C1, where the localization is based on object segmentation algorithms, such as adaptive thresholding, background subtraction, floodfill, mean shift, watershed, etc.
   C7. The system of embodiment C1, where the localization is combined with detection to produce the detection results along with the location of each possible candidates of analytes.
   C8. The system of embodiment C1, where the classification is based on deep learning, such as convolutional neural networks.
   Device and Assay with High Uniformity

### Flat Top of Pillar Spacers

In certain embodiments of the present invention, the spacers are pillars that have a flat top and a foot fixed on one plate, wherein the flat top has a smoothness with a small surface variation, and the variation is less than 5, 10 nm, 20 nm, 30 nm, 50 nm, 100 nm, 200 nm, 300 nm, 400 nm, 500 nm, 600 nm, 700 nm, 800 nm, 1000 nm, or in a range between any two of the values. A preferred flat pillar top smoothness is that surface variation of 50 nm or less.

Furthermore, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%.

### Sidewall Angle of Pillar Spacers

In certain embodiments of the present invention, the spacers are pillars that have a sidewall angle. In some embodiments, the sidewall angle is less than 5 degree (measured from the normal of a surface), 10 degree, 20 degree, 30 degree, 40 degree, 50 degree, 70 degree, or in a range between any two of the values. In a preferred embodiment, the sidewall angle is less 5 degree, 10 degree, or 20 degree.

### Formation of Uniform Thin Fluidic Layer by an Imprecise Force Pressing

In certain embodiment of the present invention, a uniform thin fluidic sample layer is formed by using a pressing with an imprecise force. The term “imprecise pressing force” without adding the details and then adding a definition for imprecise pressing force. As used herein, the term “imprecise” in the context of a force (e.g. “imprecise pressing force”) refers to a force that (a) has a magnitude that is not precisely known or precisely predictable at the time the force is applied; (b) has a pressure in the range of 0.01 kg/cm² (centimeter square) to 100 kg/cm², (c) varies in magnitude from one application of the force to the next; and (d) the imprecision (i.e. the variation) of the force in (a) and (c) is at least 20% of the total force that actually is applied.

An imprecise force can be applied by human hand, for example, e.g., by pinching an object together between a thumb and index finger, or by pinching and rubbing an object together between a thumb and index finger.

In some embodiments, the imprecise force by the hand pressing has a pressure of 0.01 kg/cm2, 0.1 kg/cm2, 0.5 kg/cm2, 1 kg/cm2, 2 kg/cm2, kg/cm2, 5 kg/cm2, 10 kg/cm2, 20 kg/cm2, 30 kg/cm2, 40 kg/cm2, 50 kg/cm2, 60 kg/cm2, 100 kg/cm2, 150 kg/cm2, 200 kg/cm2, or a range between any two of the values; and a preferred range of 0.1 kg/cm2 to 0.5 kg/cm2, 0.5 kg/cm2 to 1 kg/cm2, 1 kg/cm2 to 5 kg/cm2, 5 kg/cm2 to 10 kg/cm2 (Pressure).

### Spacer Filling Factor.

The term “spacer filling factor” or “filling factor” refers to the ratio of the spacer contact area to the total plate area”, wherein the spacer contact area refers, at a closed configuration, the contact area that the spacer's top surface contacts to the inner surface of a plate, and the total plate area refers the total area of the inner surface of the plate that the flat top of the spacers contact. Since there are two plates and each spacer has two contact surfaces each contacting one plate, the filling fact is the filling factor of the smallest.

For example, if the spacers are pillars with a flat top of a square shape (10 um×10 um), a nearly uniform cross-section and 2 um tall, and the spacers are periodic with a period of 100 urn, then the filing factor of the spacer is 1%. If in the above example, the foot of the pillar spacer is a square shape of 15 um×15 um, then the filling factor is still 1% by the definition.

### IDS\hat{ }4/hE

In certain embodiments of the present disclosure, a device for forming a thin fluidic sample layer with a uniform predetermined thickness by pressing can comprise a first plate. In certain embodiments of the present disclosure, a device for forming a thin fluidic sample layer with a uniform predetermined thickness by pressing can comprise a second plate. In certain embodiments of the present disclosure, a device for forming a thin fluidic sample layer with a uniform predetermined thickness by pressing can comprise spacers. In certain embodiments, the plates are movable relative to each other into different configurations. In certain embodiments, one or both plates are flexible. In certain embodiments, each of the plates comprises an inner surface that has a sample contact area for contacting a fluidic sample. In certain embodiments, each of the plates comprises, on its respective outer surface, a force area for applying a pressing force that forces the plates together. In certain embodiments, one or both of the plates comprise the spacers that are permanently fixed on the inner surface of a respective plate. In certain embodiments, the spacers have a predetermined substantially uniform height that is equal to or less than 200 microns, and a predetermined fixed inter-spacer-distance. In certain embodiments, the fourth power of the inter-spacer-distance (ISD) divided by the thickness (h) and the Young's modulus (E) of the flexible plate (ISD⁴/(hE)) is 5×10⁶ um³/GPa or less. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample is deposited in the open configuration and the plates are forced to the closed configuration by applying the pressing force on the force area; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure, a method of forming a thin fluidic sample layer with a uniform predetermined thickness by pressing can comprise depositing a fluidic sample on one or both of the plates when the plates are configured in an open configuration. In certain embodiments, the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for analyzing a fluidic sample can comprise a first plate. In certain embodiments of the present disclosure, a device for analyzing a fluidic sample can comprise a second plate. In certain embodiments of the present disclosure, a device for analyzing a fluidic sample can comprise spacers. In certain embodiments, the plates are movable relative to each other into different configurations. In certain embodiments, one or both plates are flexible. In certain embodiments, each of the plates has, on its respective inner surface, a sample contact area for contacting a fluidic sample. In certain embodiments, one or both of the plates comprise the spacers and the spacers are fixed on the inner surface of a respective plate. In certain embodiments, the spacers have a predetermined substantially uniform height that is equal to or less than 200 microns, and the inter-spacer-distance is predetermined. In certain embodiments, the Young's modulus of the spacers multiplied by the filling factor of the spacers is at least 2 MPa. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample is deposited in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure, a method of forming a thin fluidic sample layer with a uniform predetermined thickness by pressing can comprise depositing a fluidic sample on one or both of the plates when the plates are configured in an open configuration. In certain embodiments, the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing can comprise forcing the two plates into a closed configuration. In certain embodiments, at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for analyzing a fluidic sample can comprise a first plate. In certain embodiments of the present disclosure, a device for analyzing a fluidic sample can comprise a second plate. In certain embodiments, the plates are movable relative to each other into different configurations. In certain embodiments, one or both plates are flexible. In certain embodiments, each of the plates has, on its respective surface, a sample contact area for contacting a sample that contains an analyte. In certain embodiments, one or both of the plates comprise spacers that are permanently fixed to a plate within a sample contact area, wherein the spacers have a predetermined substantially uniform height and a predetermined fixed inter-spacer distance that is at least about 2 times larger than the size of the analyte, up to 200 um, and wherein at least one of the spacers is inside the sample contact area. In certain embodiments, one of the configurations is an open configuration, in which: the two plates are separated apart, the spacing between the plates is not regulated by the spacers, and the sample is deposited on one or both of the plates. In certain embodiments, another of the configurations is a closed configuration which is configured after the sample deposition in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure a method of forming a thin fluidic sample layer with a uniform predetermined thickness by pressing can comprise depositing a fluidic sample on one or both of the plates; when the plates are configured in an open configuration, wherein the open configuration is a configuration in which the two plates are partially or completely separated apart and the spacing between the plates is not regulated by the spacers. In certain embodiments of the present disclosure a method of forming a thin fluidic sample layer with a uniform predetermined thickness by pressing can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for forming a thin fluidic sample layer with a uniform predetermined thickness by pressing can comprise a first plate. In certain embodiments of the present disclosure, a device for forming a thin fluidic sample layer with a uniform predetermined thickness by pressing can comprise a second plate. In certain embodiments of the present disclosure, a device for forming a thin fluidic sample layer with a uniform predetermined thickness by pressing can comprise spacers. In certain embodiments, the plates are movable relative to each other into different configurations. In certain embodiments, one or both plates are flexible. In certain embodiments, each of the plates comprises, on its respective inner surface, a sample contact area for contacting and/or compressing a fluidic sample. In certain embodiments, each of the plates comprises, on its respective outer surface, an area for applying a force that forces the plates together. In certain embodiments, one or both of the plates comprise the spacers that are permanently fixed on the inner surface of a respective plate. In certain embodiments, the spacers have a predetermined substantially uniform height that is equal to or less than 200 microns, a predetermined width, and a predetermined fixed inter-spacer-distance. In certain embodiments, a ratio of the inter-spacer-distance to the spacer width is 1.5 or larger. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments of the present disclosure, a method of forming a thin fluidic sample layer with a uniform predetermined thickness by pressing with an imprecise pressing force can comprise obtaining a device of the present disclosure. In certain embodiments of the present disclosure, a method of forming a thin fluidic sample layer with a uniform predetermined thickness by pressing with an imprecise pressing force can comprise obtaining a fluidic sample. In certain embodiments of the present disclosure, a method of forming a thin fluidic sample layer with a uniform predetermined thickness by pressing with an imprecise pressing force can comprise depositing the sample on one or both of the plates; when the plates are configured in an open configuration, wherein the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing with an imprecise pressing force can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments, the spacers have a shape of pillar with a foot fixed on one of the plates and a flat top surface for contacting the other plate. In certain embodiments, the spacers have a shape of pillar with a foot fixed on one of the plates, a flat top surface for contacting the other plate, substantially uniform cross-section. In certain embodiments, the spacers have a shape of pillar with a foot fixed on one of the plates and a flat top surface for contacting the other plate, wherein the flat top surface of the pillars has a variation in less than 10 nm. In certain embodiments, the spacers have a shape of pillar with a foot fixed on one of the plates and a flat top surface for contacting the other plate, wherein the flat top surface of the pillars has a variation in less than 50 nm. In certain embodiments, the spacers have a shape of pillar with a foot fixed on one of the plates and a flat top surface for contacting the other plate, wherein the flat top surface of the pillars has a variation in less than 50 nm. In certain embodiments, the spacers have a shape of pillar with a foot fixed on one of the plates and a flat top surface for contacting the other plate, wherein the flat top surface of the pillars has a variation in less than 10 nm, 20 nm, 30 nm, 100 nm, 200 nm, or in a range of any two of the values.

In certain embodiments, the Young's modulus of the spacers multiplied by the filling factor of the spacers is at least 2 MPa. In certain embodiments, the sample comprises an analyte and the predetermined constant inter-spacer distance is at least about 2 times larger than the size of the analyte, up to 200 um. In certain embodiments, the sample comprise an analyte, the predetermined constant inter-spacer distance is at least about 2 times larger than the size of the analyte, up to 200 um, and the Young's modulus of the spacers multiplied by the filling factor of the spacers is at least 2 MPa.

In certain embodiments, a fourth power of the inter-spacer-distance (IDS) divided by the thickness (h) and the Young's modulus (E) of the flexible plate (ISD\hat{ }4/(hE)) is 5×10\hat{ }6 um\hat{ }3/GPa or less. In certain embodiments, a fourth power of the inter-spacer-distance (IDS) divided by the thickness and the Young's modulus of the flexible plate (ISD\hat{ }4/(hE)) is 1×10\hat{ }6 um\hat{ }3/GPa or less. In certain embodiments, a fourth power of the inter-spacer-distance (IDS) divided by the thickness and the Young's modulus of the flexible plate (ISD\hat{ }4/(hE)) is 5×105 um\hat{ }3/GPa or less. In certain embodiments, the Young's modulus of the spacers multiplied by the filling factor of the spacers is at least 2 MPa, and a fourth power of the inter-spacer-distance (IDS) divided by the thickness and the Young's modulus of the flexible plate (ISD\hat{ }4/(hE)) is 1×105 um\hat{ }3/GPa or less. In certain embodiments, the Young's modulus of the spacers multiplied by the filling factor of the spacers is at least 2 MPa, and a fourth power of the inter-spacer-distance (IDS) divided by the thickness and the Young's modulus of the flexible plate (ISD\hat{ }4/(hE)) is 1×10\hat{ }4 um\hat{ }3/GPa or less. In certain embodiments, the Young's modulus of the spacers multiplied by the filling factor of the spacers is at least 20 MPa.

In certain embodiments of the present disclosure, the ratio of the inter-spacing distance of the spacers to the average width of the spacer is 2 or larger. In certain embodiments, the ratio of the inter-spacing distance of the spacers to the average width of the spacer is 2 or larger, and the Young's modulus of the spacers multiplied by the filling factor of the spacers is at least 2 MPa. In certain embodiments, the inter-spacer distance that is at least about 2 times larger than the size of the analyte, up to 200 um. In certain embodiments, a ratio of the inter-spacer-distance to the spacer width is 1.5 or larger. In certain embodiments, a ratio of the width to the height of the spacer is 1 or larger. In certain embodiments, a ratio of the width to the height of the spacer is 1.5 or larger. In certain embodiments, a ratio of the width to the height of the spacer is 2 or larger. In certain embodiments, a ratio of the width to the height of the spacer is larger than 2, 3, 5, 10, 20, 30, 50, or in a range of any two the value.

In certain embodiments, a force that presses the two plates into the closed configuration is an imprecise pressing force. In certain embodiments, a force that presses the two plates into the closed configuration is an imprecise pressing force provided by human hand. In certain embodiments, the forcing of the two plates to compress at least part of the sample into a layer of substantially uniform thickness comprises a use of a conformable pressing, either in parallel or sequentially, an area of at least one of the plates to press the plates together to a closed configuration, wherein the conformable pressing generates a substantially uniform pressure on the plates over the at least part of the sample, and the pressing spreads the at least part of the sample laterally between the sample contact surfaces of the plates, and wherein the closed configuration is a configuration in which the spacing between the plates in the layer of uniform thickness region is regulated by the spacers; and wherein the reduced thickness of the sample reduces the time for mixing the reagents on the storage site with the sample. In certain embodiments, the pressing force is an imprecise force that has a magnitude which is, at the time that the force is applied, either (a) unknown and unpredictable, or (b) cannot be known and cannot be predicted within an accuracy equal or better than 20% of the average pressing force applied. In certain embodiments, the pressing force is an imprecise force that has a magnitude which is, at the time that the force is applied, either (a) unknown and unpredictable, or (b) cannot be known and cannot be predicted within an accuracy equal or better than 30% of the average pressing force applied. In certain embodiments, the pressing force is an imprecise force that has a magnitude which is, at the time that the force is applied, either (a) unknown and unpredictable, or (b) cannot be known and cannot be predicted within an accuracy equal or better than 30% of the average pressing force applied; and wherein the layer of highly uniform thickness has a variation in thickness uniform of 20% or less. In certain embodiments, the pressing force is an imprecise force that has a magnitude which cannot, at the time that the force is applied, be determined within an accuracy equal or better than 30%, 40%, 50%, 70%, 100%, 200%, 300%, 500%, 1000%, 2000%, or in a range between any of the two values.

In certain embodiments of the present disclosure, the flexible plate has a thickness of in the range of 10 um to 200 um. In certain embodiments, the flexible plate has a thickness of in the range of 20 um to 100 um. In certain embodiments, the flexible plate has a thickness of in the range of 25 um to 180 um. In certain embodiments, the flexible plate has a thickness of in the range of 200 um to 260 um. In certain embodiments, the flexible plate has a thickness of equal to or less than 250 um, 225 um, 200 um, 175 um, 150 um, 125 um, 100 um, 75 um, 50 urn, 25 um, 10 um, 5 um, 1 um, or in a range between the two of the values. In certain embodiments, the sample has a viscosity in the range of 0.1 to 4 (mPa s). In certain embodiments, the flexible plate has a thickness of in the range of 200 um to 260 um. In certain embodiments, the flexible plate has a thickness in the range of 20 um to 200 um and Young's modulus in the range 0.1 to 5 GPa.

In certain embodiments of the present disclosure, the sample deposition is a deposition directly from a subject to the plate without using any transferring devices. In certain embodiments, during the deposition, the amount of the sample deposited on the plate is unknown. In certain embodiments, the method further comprises an analyzing that analyze the sample. In certain embodiments, the analyzing comprises calculating the volume of a relevant sample volume by measuring the lateral area of the relevant sample volume and calculating the volume from the lateral area and the predetermined spacer height. In certain embodiments, the pH value at location of a sample that is between the two plates in a closed configuration is determined by the volume of the location and by analyzing an image(s) taken from that location. In certain embodiments, the determination by analyzing an image uses artificial intelligence and machine learning.

In certain embodiments, the analyzing step (e) comprises measuring: i. imaging, ii. luminescence selected from photoluminescence, electroluminescence, and electrochemiluminescence, iii. surface Raman scattering, iv. electrical impedance selected from resistance, capacitance, and inductance, or v. any combination of i-iv. In certain embodiments, the analyzing comprises reading, image analysis, or counting of the analyte, or a combination of thereof. In certain embodiments, the sample contains one or plurality of analytes, and one or both plate sample contact surfaces comprise one or a plurality of binding sites that each binds and immobilize a respective analyte. In certain embodiments, one or both plate sample contact surfaces comprise one or a plurality of storage sites that each stores a reagent or reagents, wherein the reagent(s) dissolve and diffuse in the sample. In certain embodiments, one or both plate sample contact surfaces comprises one or a plurality of amplification sites that are each capable of amplifying a signal from the analyte or a label of the analyte when the analyte or label is within 500 nm from an amplification site. In certain embodiments, i. one or both plate sample contact surfaces comprise one or a plurality of binding sites that each binds and immobilize a respective analyte; or ii. one or both plate sample contact surfaces comprise, one or a plurality of storage sites that each stores a reagent or reagents; wherein the reagent(s) dissolve and diffuse in the sample, and wherein the sample contains one or plurality of analytes; or iii. one or a plurality of amplification sites that are each capable of amplifying a signal from the analyte or a label of the analyte when the analyte or label is 500 nm from the amplification site; or iv. any combination of i to iii.

In certain embodiments, the liquid sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

In certain embodiments, the layer of uniform thickness in the closed configuration is less than 150 um. In certain embodiments, the pressing is provided by a pressured liquid, a pressed gas, or a conformal material. In certain embodiments, the analyzing comprises counting cells in the layer of uniform thickness. In certain embodiments, the analyzing comprises performing an assay in the layer of uniform thickness. In certain embodiments. In certain embodiments, the assay is a binding assay or biochemical assay. In certain embodiments, the sample deposited has a total volume less 0.5 uL. In certain embodiments, multiple drops of sample are deposited onto one or both of the plates.

In certain embodiments, the inter-spacer distance is in the range of 1 μm to 120 μm. In certain embodiments, the inter-spacer distance is in the range of 120 μm to 50 μm. In certain embodiments, the inter-spacer distance is in the range of 120 μm to 200 μm. In certain embodiments, the flexible plates have a thickness in the range of 20 um to 250 um and Young's modulus in the range 0.1 to 5 GPa. In certain embodiments, for a flexible plate, the thickness of the flexible plate times the Young's modulus of the flexible plate is in the range 60 to 750 GPa-um.

In certain embodiments, the layer of uniform thickness sample is uniform over a lateral area that is at least 1 mm². In certain embodiments, the layer of uniform thickness sample is uniform over a lateral area that is at least 3 mm². In certain embodiments, the layer of uniform thickness sample is uniform over a lateral area that is at least 5 mm². In certain embodiments. In certain embodiments, the layer of uniform thickness sample is uniform over a lateral area that is at least 10 mm². In certain embodiments, the layer of uniform thickness sample is uniform over a lateral area that is at least 20 mm². In certain embodiments, the layer of uniform thickness sample is uniform over a lateral area that is in a range of 20 mm² to 100 mm². In certain embodiments, the layer of uniform thickness sample has a thickness uniformity of up to +/−5% or better. In certain embodiments, the layer of uniform thickness sample has a thickness uniformity of up to +1-10% or better. In certain embodiments, the layer of uniform thickness sample has a thickness uniformity of up to +1-20% or better. In certain embodiments, the layer of uniform thickness sample has a thickness uniformity of up to +/−30% or better. In certain embodiments, the layer of uniform thickness sample has a thickness uniformity of up to +/−40% or better. In certain embodiments, the layer of uniform thickness sample has a thickness uniformity of up to +/−50% or better.

In certain embodiments, the spacers are pillars with a cross-sectional shape selected from round, polygonal, circular, square, rectangular, oval, elliptical, or any combination of the same. In certain embodiments, the spacers have pillar shape, have a substantially flat top surface, and have substantially uniform cross-section, wherein, for each spacer, the ratio of the lateral dimension of the spacer to its height is at least 1. In certain embodiments, the inter spacer distance is periodic. In certain embodiments, the spacers have a filling factor of 1% or higher, wherein the filling factor is the ratio of the spacer contact area to the total plate area. In certain embodiments, the Young's modulus of the spacers times the filling factor of the spacers is equal or larger than 20 MPa, wherein the filling factor is the ratio of the spacer contact area to the total plate area. In certain embodiments, the spacing between the two plates at the closed configuration is in less 200 um. In certain embodiments, the spacing between the two plates at the closed configuration is a value selected from between 1.8 um and 3.5 um. In certain embodiments, the spacing are fixed on a plate by directly embossing the plate or injection molding of the plate. In certain embodiments, the materials of the plate and the spacers are selected from polystyrene, PMMA, PC, COC, COP, or another plastic. In certain embodiments, the spacers have a pillar shape, and the sidewall corners of the spacers have a round shape with a radius of curvature at least 1 μm. In certain embodiments, the spacers have a density of at least 1000/mm². In certain embodiments, at least one of the plates is transparent. In certain embodiments, the mold used to make the spacers is fabricated by a mold containing features that are fabricated by either (a) directly reactive ion etching or ion beam etched or (b) by a duplication or multiple duplication of the features that are reactive ion etched or ion beam etched.

In certain embodiments, the spacers are configured, such that the filling factor is in the range of 1% to 5%. In certain embodiments, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%. In certain embodiments, the spacers are configured, such that the filling factor is in the range of 1% to 5%. In certain embodiments, the spacers are configured, such that the filling factor is in the range of 5% to 10%. In certain embodiments, the spacers are configured, such that the filling factor is in the range of 10% to 20%. In certain embodiments, the spacers are configured, such that the filling factor is in the range of 20% to 30%. In certain embodiments, the spacers are configured, such that the filling factor is 5%, 10%, 20%, 30%, 40%, 50%, or in a range of any two of the values. In certain embodiments, the spacers are configured, such that the filling factor is 50%, 60%, 70%, 80%, or in a range of any two of the values.

In certain embodiments, the spacers are configured, such that the filling factor multiplies the Young's modulus of the spacer is in the range of 2 MPa and 10 MPa. In certain embodiments, the spacers are configured, such that the filling factor multiplies the Young's modulus of the spacer is in the range of 10 MPa and 20 MPa. In certain embodiments, the spacers are configured, such that the filling factor multiplies the Young's modulus of the spacer is in the range of 20 MPa and 40 MPa. In certain embodiments, the spacers are configured, such that the filling factor multiplies the Young's modulus of the spacer is in the range of 40 MPa and 80 MPa. In certain embodiments, the spacers are configured, such that the filling factor multiplies the Young's modulus of the spacer is in the range of 80 MPa and 120 MPa. In certain embodiments, the spacers are configured, such that the filling factor multiplies the Young's modulus of the spacer is in the range of 120 MPa to 150 MPa.

In certain embodiments, the device further comprises a dry reagent coated on one or both plates. In certain embodiments, the device further comprises, on one or both plates, a dry binding site that has a predetermined area, wherein the dry binding site binds to and immobilizes an analyte in the sample. In certain embodiments, the device further comprises, on one or both plates, a releasable dry reagent and a release time control material that delays the time that the releasable dry regent is released into the sample. In certain embodiments, the release time control material delays the time that the dry regent starts is released into the sample by at least 3 seconds. In certain embodiments, the regent comprises anticoagulant and/or staining reagent(s). In certain embodiments, the reagent comprises cell lysing reagent(s). In certain embodiments, the device further comprises, on one or both plates, one or a plurality of dry binding sites and/or one or a plurality of reagent sites. In certain embodiments, the analyte comprises a molecule (e.g., a protein, peptides, DNA, RNA, nucleic acid, or other molecule), cells, tissues, viruses, and nanoparticles with different shapes. In certain embodiments, the analyte comprises white blood cells, red blood cells and platelets. In certain embodiments, the analyte is stained.

In certain embodiments, the spacers regulating the layer of uniform thickness have a filling factor of at least 1%, wherein the filling factor is the ratio of the spacer area in contact with the layer of uniform thickness to the total plate area in contact with the layer of uniform thickness. In certain embodiments, for spacers regulating the layer of uniform thickness, the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness. In certain embodiments, for a flexible plate, the thickness of the flexible plate times the Young's modulus of the flexible plate is in the range 60 to 750 GPa-um. In certain embodiments, for a flexible plate, the fourth power of the inter-spacer-distance (ISD) divided by the thickness of the flexible plate (h) and the Young's modulus (E) of the flexible plate, ISD⁴/(hE), is equal to or less than 10⁶ um³/GPa.

In certain embodiments, one or both plates comprises a location marker, either on a surface of or inside the plate, that provide information of a location of the plate. In certain embodiments, one or both plates comprises a scale marker, either on a surface of or inside the plate, that provide information of a lateral dimension of a structure of the sample and/or the plate. In certain embodiments, one or both plates comprises an imaging marker, either on surface of or inside the plate, that assists an imaging of the sample. In certain embodiments, the spacers functions as a location marker, a scale marker, an imaging marker, or any combination of thereof.

In certain embodiments, the average thickness of the layer of uniform thickness is about equal to a minimum dimension of an analyte in the sample. In certain embodiments, the inter-spacer distance is in the range of 7 μm to 50 μm. In certain embodiments, the inter-spacer distance is in the range of 50 μm to 120 μm. In certain embodiments, the inter-spacer distance is in the range of 120 μm to 200 μm (micron). In certain embodiments, the inter-spacer distance is substantially periodic. In certain embodiments, the spacers are pillars with a cross-sectional shape selected from round, polygonal, circular, square, rectangular, oval, elliptical, or any combination of the same.

In certain embodiments, the spacers have a pillar shape and have a substantially flat top surface, wherein, for each spacer, the ratio of the lateral dimension of the spacer to its height is at least 1. In certain embodiments, each spacer has the ratio of the lateral dimension of the spacer to its height is at least 1. In certain embodiments, the minimum lateral dimension of spacer is less than or substantially equal to the minimum dimension of an analyte in the sample. In certain embodiments, the minimum lateral dimension of spacer is in the range of 0.5 um to 100 um. In certain embodiments, the minimum lateral dimension of spacer is in the range of 0.5 um to 10 um.

In certain embodiments, the sample is blood. In certain embodiments, the sample is whole blood without dilution by liquid. In certain embodiments, the sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine. In certain embodiments, the sample is a biological sample, an environmental sample, a chemical sample, or clinical sample.

In certain embodiments, the spacers have a pillar shape, and the sidewall corners of the spacers have a round shape with a radius of curvature at least 1 μm. In certain embodiments, the spacers have a density of at least 100/mm². In certain embodiments, the spacers have a density of at least 1000/mm². In certain embodiments, at least one of the plates is transparent. In certain embodiments, at least one of the plates is made from a flexible polymer. In certain embodiments, for a pressure that compresses the plates, the spacers are not compressible and/or, independently, only one of the plates is flexible. In certain embodiments, the flexible plate has a thickness in the range of 10 um to 200 um. In certain embodiments, the variation is less than 30%. In certain embodiments, the variation is less than 10%. In certain embodiments, the variation is less than 5%.

In certain embodiments, the first and second plates are connected and are configured to be changed from the open configuration to the closed configuration by folding the plates. In certain embodiments, the first and second plates are connected by a hinge and are configured to be changed from the open configuration to the closed configuration by folding the plates along the hinge. In certain embodiments, the first and second plates are connected by a hinge that is a separate material to the plates, and are configured to be changed from the open configuration to the closed configuration by folding the plates along the hinge. In certain embodiments, the first and second plates are made in a single piece of material and are configured to be changed from the open configuration to the closed configuration by folding the plates. In certain embodiments, the layer of uniform thickness sample is uniform over a lateral area that is at least 1 mm².

In certain embodiments, the device is configured to analyze the sample in 60 seconds or less. In certain embodiments, at the closed configuration, the final sample thickness device is configured to analyze the sample in 60 seconds or less. In certain embodiments, at the closed configuration, the final sample thickness device is configured to analyze the sample in 10 seconds or less.

In certain embodiments, the dry binding site comprises a capture agent. In certain embodiments, the dry binding site comprises an antibody or nucleic acid. In certain embodiments, the releasable dry reagent is a labeled reagent. In certain embodiments, the releasable dry reagent is a fluorescently-labeled reagent. In certain embodiments, the releasable dry reagent is a fluorescently-labeled antibody. In certain embodiments, the releasable dry reagent is a cell stain. In certain embodiments, the releasable dry reagent is a cell lysing.

In certain embodiments, the detector is an optical detector that detects an optical signal. In certain embodiments, the detector is an electric detector that detect electrical signal. In certain embodiments, the spacing are fixed on a plate by directly embossing the plate or injection molding of the plate. In certain embodiments, the materials of the plate and the spacers are selected from polystyrene, PMMA, PC, COC, COP, or another plastic.

In certain embodiments of the present disclosure, a system for rapidly analyzing a sample using a mobile phone can comprise a device of any prior embodiment. In certain embodiments of the present disclosure, a system for rapidly analyzing a sample using a mobile phone can comprise a mobile communication device. In certain embodiments, the mobile communication device can comprise one or a plurality of cameras for the detecting and/or imaging the sample. In certain embodiments, the mobile communication device can comprise electronics, signal processors, hardware and software for receiving and/or processing the detected signal and/or the image of the sample and for remote communication. In certain embodiments, the mobile communication device can comprise a light source from either the mobile communication device or an external source. In same embodiments, the detector in the devices or methods of any prior embodiment is provided by the mobile communication device, and detects an analyte in the sample at the closed configuration.

In certain embodiments, one of the plates has a binding site that binds an analyte, wherein at least part of the uniform sample thickness layer is over the binding site, and is substantially less than the average lateral linear dimension of the binding site. In certain embodiments, any system of the present disclosure can comprise a housing configured to hold the sample and to be mounted to the mobile communication device. In certain embodiments, the housing comprises optics for facilitating the imaging and/or signal processing of the sample by the mobile communication device, and a mount configured to hold the optics on the mobile communication device. In certain embodiments, an element of the optics in the housing is movable relative to the housing. In certain embodiments, the mobile communication device is configured to communicate test results to a medical professional, a medical facility or an insurance company. In certain embodiments, the mobile communication device is further configured to communicate information on the test and the subject with the medical professional, medical facility or insurance company. In certain embodiments, the mobile communication device is further configured to communicate information of the test to a cloud network, and the cloud network process the information to refine the test results. In certain embodiments, the mobile communication device is further configured to communicate information of the test and the subject to a cloud network, the cloud network process the information to refine the test results, and the refined test results will send back the subject. In certain embodiments, the mobile communication device is configured to receive a prescription, diagnosis or a recommendation from a medical professional. In certain embodiments, the mobile communication device is configured with hardware and software to capture an image of the sample. In certain embodiments, the mobile communication device is configured with hardware and software to analyze a test location and a control location in in image. In certain embodiments, the mobile communication device is configured with hardware and software to compare a value obtained from analysis of the test location to a threshold value that characterizes the rapid diagnostic test.

In certain embodiments of the present disclosure, at least one of the plates comprises a storage site in which assay reagents are stored. In certain embodiments, at least one of the cameras reads a signal from the device. In certain embodiments, the mobile communication device communicates with the remote location via a wifi or cellular network. In certain embodiments, the mobile communication device is a mobile phone.

In certain embodiments of the present disclosure, a method for rapidly analyzing an analyte in a sample using a mobile phone can comprise depositing a sample on the device of any prior system embodiment. In certain embodiments of the present disclosure, a method for rapidly analyzing an analyte in a sample using a mobile phone can comprise assaying an analyte in the sample deposited on the device to generate a result. In certain embodiments of the present disclosure, a method for rapidly analyzing an analyte in a sample using a mobile phone can comprise communicating the result from the mobile communication device to a location remote from the mobile communication device.

In certain embodiments, the analyte comprises a molecule (e.g., a protein, peptides, DNA, RNA, nucleic acid, or other molecule), cells, tissues, viruses, and nanoparticles with different shapes. In certain embodiments, the analyte comprises white blood cell, red blood cell and platelets. In certain embodiments, the assaying comprises performing a white blood cells differential assay. In certain embodiments, a method of the present disclosure can comprise analyzing the results at the remote location to provide an analyzed result. In certain embodiments, a method of the present disclosure can comprise communicating the analyzed result from the remote location to the mobile communication device. In certain embodiments, the analysis is done by a medical professional at a remote location. In certain embodiments, the mobile communication device receives a prescription, diagnosis or a recommendation from a medical professional at a remote location.

In certain embodiments, the sample is a bodily fluid. In certain embodiments, the bodily fluid is blood, saliva or urine. In certain embodiments, the sample is whole blood without dilution by a liquid. In certain embodiments, the assaying step comprises detecting an analyte in the sample. In certain embodiments, the analyte is a biomarker. In certain embodiments, the analyte is a protein, nucleic acid, cell, or metabolite. In certain embodiments, the method comprises counting the number of red blood cells. In certain embodiments, the method comprises counting the number of white blood cells. In certain embodiments, the method comprises staining the cells in the sample and counting the number of neutrophils, lymphocytes, monocytes, eosinophils and basophils. In certain embodiments, the assay done in step (b) is a binding assay or a biochemical assay.

In certain embodiments of the present disclosure, a method for analyzing a sample can comprise obtaining a device of any prior device embodiment. In certain embodiments of the present disclosure, a method for analyzing a sample can comprise depositing the sample onto one or both pates of the device. In certain embodiments of the present disclosure, a method for analyzing a sample can comprise placing the plates in a closed configuration and applying an external force over at least part of the plates. In certain embodiments of the present disclosure, a method for analyzing a sample can comprise analyzing the layer of uniform thickness while the plates are the closed configuration.

In certain embodiments, the first plate further comprises, on its surface, a first predetermined assay site and a second predetermined assay site, wherein the distance between the edges of the assay site is substantially larger than the thickness of the uniform thickness layer when the plates are in the closed position, wherein at least a part of the uniform thickness layer is over the predetermined assay sites, and wherein the sample has one or a plurality of analytes that are capable of diffusing in the sample. In certain embodiments, the first plate has, on its surface, at least three analyte assay sites, and the distance between the edges of any two neighboring assay sites is substantially larger than the thickness of the uniform thickness layer when the plates are in the closed position, wherein at least a part of the uniform thickness layer is over the assay sites, and wherein the sample has one or a plurality of analytes that are capable of diffusing in the sample. In certain embodiments, the first plate has, on its surface, at least two neighboring analyte assay sites that are not separated by a distance that is substantially larger than the thickness of the uniform thickness layer when the plates are in the closed position, wherein at least a part of the uniform thickness layer is over the assay sites, and wherein the sample has one or a plurality of analytes that are capable of diffusing in the sample. In certain embodiments, the analyte assay area is between a pair of electrodes. In certain embodiments, the assay area is defined by a patch of dried reagent. In certain embodiments, the assay area binds to and immobilizes the analyte. In certain embodiments, the assay area is defined by a patch of binding reagent that, upon contacting the sample, dissolves into the sample, diffuses in the sample, and binds to the analyte. In certain embodiments, the inter-spacer distance is in the range of 14 μm to 200 μm. In certain embodiments, the inter-spacer distance is in the range of 7 μm to 20 μm. In certain embodiments, the spacers are pillars with a cross-sectional shape selected from round, polygonal, circular, square, rectangular, oval, elliptical, or any combination of the same. In certain embodiments, the spacers have a pillar shape and have a substantially flat top surface, wherein, for each spacer, the ratio of the lateral dimension of the spacer to its height is at least 1. In certain embodiments, the spacers have a pillar shape, and the sidewall corners of the spacers have a round shape with a radius of curvature at least 1 μm. In certain embodiments, the spacers have a density of at least 1000/mm². In certain embodiments, at least one of the plates is transparent. In certain embodiments, at least one of the plates is made from a flexible polymer. In certain embodiments, only one of the plates is flexible. In certain embodiments, the area-determination device is a camera. In certain embodiments, an area in the sample contact area of a plate, wherein the area is less than 1/100, 1/20, 1/10, ⅙, ⅕, ¼, ⅓, ½, ⅔ of the sample contact area, or in a range between any of the two values. In certain embodiments, the area-determination device comprises a camera and an area in the sample contact area of a plate, wherein the area is in contact with the sample.

In certain embodiments, the deformable sample comprises a liquid sample. In certain embodiments, the imprecision force has a variation at least 30% of the total force that actually is applied. In certain embodiments, the imprecision force has a variation at least 20%, 30%, 40%, 50%, 60, 70%, 80%, 90% 100%, 150%, 200%, 300%, 500%, or in a range of any two values, of the total force that actually is applied. In certain embodiments, the spacers have a flat top. In certain embodiments, the device is further configured to have, after the pressing force is removed, a sample thickness that is substantially the same in thickness and uniformity as that when the force is applied. In certain embodiments, the imprecise force is provided by human hand. In certain embodiments, the inter spacer distance is substantially constant. In certain embodiments, the inter spacer distance is substantially periodic in the area of the uniform sample thickness area. In certain embodiments, the multiplication product of the filling factor and the Young's modulus of the spacer is 2 MPa or larger. In certain embodiments, the force is applied by hand directly or indirectly. In certain embodiments, the force applied is in the range of 1 N to 20 N. In certain embodiments, the force applied is in the range of 20 N to 200 N. In certain embodiments, the highly uniform layer has a thickness that varies by less than 15%, 10%, or 5% of an average thickness. In certain embodiments, the imprecise force is applied by pinching the device between a thumb and forefinger. In certain embodiments, the predetermined sample thickness is larger than the spacer height. In certain embodiments, the device holds itself in the closed configuration after the pressing force has been removed. In certain embodiments, the uniform thickness sample layer area is larger than that area upon which the pressing force is applied. In certain embodiments, the spacers do not significantly deform during application of the pressing force. In certain embodiments, the pressing force is not predetermined beforehand and is not measured. In certain embodiments, the fluidic sample is replaced by a deformable sample and the embodiments for making at least a part of the fluidic sample into a uniform thickness layer can make at least a part of the deformable sample into a uniform thickness layer. In certain embodiments, the inter spacer distance is periodic. In certain embodiments, the spacers have a flat top. In certain embodiments, the inter spacer distance is at least two times large than the size of the targeted analyte in the sample.

### Manufacturing of Q-Card

In certain embodiments of the present disclosure, a Q-Card can comprise a first plate. In certain embodiments of the present disclosure, a Q-Card can comprise a second plate. In certain embodiments of the present disclosure, a Q-Card can comprise a hinge. In certain embodiments, the first plate, that is about 200 nm to 1500 nm thick, comprises, on its inner surface, (a) a sample contact area for contacting a sample, and (b) a sample overflow dam that surrounds the sample contact area is configured to present a sample flow outside of the dam. In certain embodiments, the second plate is 10 um to 250 um thick and comprises, on its inner surface, (a) a sample contact area for contacting a sample, and (b) spacers on the sample contact area. In certain embodiments, the hinge that connect the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, (a) a sample contact area for contacting a sample, (b) a sample overflow dam that surrounds the sample contact area is configured to present a sample flow outside of the dam, and (c) spacers on the sample contact area. In certain embodiments, the second plate, that is 10 um to 250 um thick, comprises, on its inner surface, a sample contact area for contacting a sample. In certain embodiments, the hinge connects the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, (a) a sample contact area for contacting a sample, and (b) spacers on the sample contact area. In certain embodiments, the second plate, that is 10 um to 250 um thick, comprises, on its inner surface, (a) a sample contact area for contacting a sample, and (b) a sample overflow dam that surrounds the sample contact area is configured to present a sample flow outside of the dam. In certain embodiments, the hinge connects the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, a sample contact area for contacting a sample. In certain embodiments, the second plate, that is 10 um to 250 um thick, comprises, on its inner surface, (a) a sample contact area for contacting a sample, (b) a sample overflow dam that surrounds the sample contact area is configured to present a sample flow outside of the dam, and (c) spacers on the sample contact area. In certain embodiments, the hinge connects the first and the second plates. In certain embodiments, the first and second plate are movable relative to each other around the axis of the hinge.

In certain embodiments of the present disclosure, a method for fabricating any Q-Card of the present disclosure can comprise injection molding of the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise Laser cutting the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise injection molding and laser cutting the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise nanoimprinting or extrusion printing to fabricated both the first and the second plate.

In certain embodiments of the present disclosure, a method for fabricating any Q-Card of the present disclosure can comprise fabricating the first plate or the second plate, using injection molding, laser cutting the first plate, nanoimprinting, extrusion printing, or a combination of thereof.

In certain embodiments of the present disclosure, a method for fabricating any Q-Card of the present disclosure can comprise a step of attaching the hinge on the first and the second plates after the fabrication of the first and second plates.

### Compressed Regulated Open Flow” (CROF)

In assaying, a manipulation of a sample or a reagent can lead to improvements in the assaying. The manipulation includes, but not limited to, manipulating the geometric shape and location of a sample and/or a reagent, a mixing or a binding of a sample and a reagent, and a contact area of a sample of reagent to a plate.

Many embodiments of the present invention manipulate the geometric size, location, contact areas, and mixing of a sample and/or a reagent using a method, termed “compressed regulated open flow (CROF)”, and a device that performs CROF.

The term “compressed open flow (COF)” refers to a method that changes the shape of a flowable sample deposited on a plate by (i) placing other plate on top of at least a part of the sample and (ii) then compressing the sample between two plates by pushing the two plates towards each other; wherein the compression reduces a thickness of at least a part of the sample and makes the sample flow into open spaces between the plates.

The term “compressed regulated open flow” or “CROF” (or “self-calibrated compressed open flow” or “SCOF” or “SCCOF”) refers to a particular type of COF, wherein the final thickness of a part or entire sample after the compression is “regulated” by spacers, wherein the spacers, that are placed between the two plates.

The term “the final thickness of a part or entire sample is regulated by spacers” in a CROF means that during a CROF, once a specific sample thickness is reached, the relative movement of the two plates and hence the change of sample thickness stop, wherein the specific thickness is determined by the spacer.

One embodiment of the method of CROF, as illustrated in FIG. 7, comprises:

(a) obtaining a sample, that is flowable;

(b) obtaining a first plate and a second plate that are movable relative to each other into different configurations, wherein each plate has a sample contact surface that is substantially planar, wherein one or both of the plates comprise spacers and the spacers have a predetermined height, and the spacers are on a respective sample contacting surface;

(c) depositing, when the plates are configured in an open configuration, the sample on one or both of the plates; wherein the open configuration is a configuration in which the two plates are either partially or completely separated apart and the spacing between the plates is not regulated by the spacers; and

(d) after (c), spreading the sample by bringing the plates into a closed configuration, wherein, in the closed configuration: the plates are facing each other, the spacers and a relevant volume of the sample are between the plates, the thickness of the relevant volume of the sample is regulated by the plates and the spacers, wherein the relevant volume is at least a portion of an entire volume of the sample, and wherein during the sample spreading, the sample flows laterally between the two plates.

The term “plate” refers to, unless being specified otherwise, the plate used in a CROF process, which a solid that has a surface that can be used, together with another plate, to compress a sample placed between the two plate to reduce a thickness of the sample.

The term “the plates” or “the pair of the plates” refers to the two plates in a CROF process.

The term “first plate” or “second plate” refers to the plate use in a CROF process.

The term “the plates are facing each other” refers to the cases where a pair of plates are at least partially facing each other.

The term “spacers” or “stoppers” refers to, unless stated otherwise, the mechanical objects that set, when being placed between two plates, a limit on the minimum spacing between the two plates that can be reached when compressing the two plates together. Namely, in the compressing, the spacers will stop the relative movement of the two plates to prevent the plate spacing becoming less than a preset (i.e. predetermined) value. There are two types of the spacers: “open-spacers” and “enclosed-spacers”.

The term “open-spacer” means the spacer have a shape that allows a liquid to flow around the entire perimeter of the spacer and flow pass the spacer. For example, a pillar is an open spacer.

The term of “enclosed spacer” means the spacer of having a shape that a liquid cannot flow abound the entire perimeter of the spacer and cannot flow pass the spacer. For example, a ring shape spacer is an enclosed spacer for a liquid inside the ring, where the liquid inside the ring spacer remains inside the ring and cannot go to outside (outside perimeter).

The term “a spacer has a predetermined height” and “spacers have predetermined inter-spacer distance” means, respectively, that the value of the spacer height and the inter spacer distance is known prior to a CROF process. It is not predetermined, if the value of the spacer height and the inter-spacer distance is not known prior to a CROF process. For example, in the case that beads are sprayed on a plate as spacers, where beads are landed on random locations of the plate, the inter-spacer distance is not predetermined. Another example of not predetermined inter spacer distance is that the spacers moves during a CROF processes.

The term “a spacer is fixed on its respective plate” in a CROF process means that the spacer is attached to a location of a plate and the attachment to that location is maintained during a CROF (i.e. the location of the spacer on respective plate does not change). An example of “a spacer is fixed with its respective plate” is that a spacer is monolithically made of one piece of material of the plate, and the location of the spacer relative to the plate surface does not change during CROF. An example of “a spacer is not fixed with its respective plate” is that a spacer is glued to a plate by an adhesive, but during a use of the plate, during CROF, the adhesive cannot hold the spacer at its original location on the plate surface and the spacer moves away from its original location on the plate surface.

The term “a spacer is fixed to a plate monolithically” means the spacer and the plate behavior like a single piece of an object where, during a use, the spacer does not move or separated from its original location on the plate.

The term “open configuration” of the two plates in a CROF process means a configuration in which the two plates are either partially or completely separated apart and the spacing between the plates is not regulated by the spacers

The term “closed configuration” of the two plates in a CROF process means a configuration in which the plates are facing each other, the spacers and a relevant volume of the sample are between the plates, the thickness of the relevant volume of the sample is regulated by the plates and the spacers, wherein the relevant volume is at least a portion of an entire volume of the sample.

The term “a sample thickness is regulated by the plate and the spacers” in a CROF process means that for a give condition of the plates, the sample, the spacer, and the plate compressing method, the thickness of at least a port of the sample at the closed configuration of the plates can be predetermined from the properties of the spacers and the plate.

The term “inner surface” or “sample surface” of a plate in a CROF device refers to the surface of the plate that touches the sample, while the other surface (that does not touch the sample) of the plate is termed “outer surface”.

The term “X-Plate” of a CROF device refers to a plate that comprises spaces that are on the sample surface of the plate, wherein the spacers have a predetermined inter-spacer distance and spacer height, and wherein at least one of the spacers is inside the sample contact area.

The term “CROF device” refers to a device that performs a CROF process. The term “CROFed” means that a CROF process is used. For example, the term “a sample was CROFed” means that the sample was put inside a CROF device, a CROF process was performed, and the sample was hold, unless stated otherwise, at a final configuration of the CROF.

The term “CROF plates” refers to the two plates used in performing a CROF process. The term “surface smoothness” or “surface smoothness variation” of a planar surface refers to the average deviation of a planar surface from a perfect flat plane over a short distance that is about or smaller than a few micrometers. The surface smoothness is different from the surface flatness variation. A planar surface can have a good surface flatness, but poor surface smoothness.

The term “surface flatness” or “surface flatness variation” of a planar surface refers to the average deviation of a planar surface from a perfect flat plane over a long distance that is about or larger than 10 um. The surface flatness variation is different from the surface smoothness. A planar surface can have a good surface smoothness, but poor surface flatness (i.e. large surface flatness variation).

The term “relative surface flatness” of a plate or a sample is the ratio of the plate surface flatness variation to the final sample thickness.

The term “final sample thickness” in a CROF process refers to, unless specified otherwise, the thickness of the sample at the closed configuration of the plates in a CORF process.

The term “compression method” in CROF refers to a method that brings two plates from an open configuration to a closed configuration.

The term of “interested area” or “area of interest” of a plate refers to the area of the plate that is relevant to the function that the plates perform.

The term “at most” means “equal to or less than”. For example, a spacer height is at most 1 um, it means that the spacer height is equal to or less than 1 um.

The term “sample area” means the area of the sample in the direction approximately parallel to the space between the plates and perpendicular to the sample thickness.

The term “sample thickness” refers to the sample dimension in the direction normal to the surface of the plates that face each other (e.g., the direction of the spacing between the plates).

The term “plate-spacing” refers to the distance between the inner surfaces of the two plates.

The term “deviation of the final sample thickness” in a CROF means the difference between the predetermined spacer height (determined from fabrication of the spacer) and the average of the final sample thickness, wherein the average final sample thickness is averaged over a given area (e.g. an average of 25 different points (4 mm apart) over 1.6 cm by 1.6 cm area).

The term “uniformity of the measured final sample thickness” in a CROF process means the standard deviation of the measured final sample thickness over a given sample area (e.g. the standard deviation relative to the average.).

The term “relevant volume of a sample” and “relevant area of a sample” in a CROF process refers to, respectively, the volume and the area of a portion or entire volume of the sample deposited on the plates during a CROF process, that is relevant to a function to be performed by a respective method or device, wherein the function includes, but not limited to, reduction in binding time of analyte or entity, detection of analytes, quantify of a volume, quantify of a concentration, mixing of reagents, or control of a concentration (analytes, entity or reagents).

The term “some embodiments”, “in some embodiments” “in the present invention, in some embodiments”, “embodiment”, “one embodiment”, “another embodiment”, “certain embodiments”, “many embodiments”, or alike refers, unless specifically stated otherwise, to an embodiment(s) that is (are) applied to the entire disclosure (i.e. the entire invention).

The term “height” or “thickness” of an object in a CROF process refers to, unless specifically stated, the dimension of the object that is in the direction normal to a surface of the plate. For example, spacer height is the dimension of the spacer in the direction normal to a surface of the plate, and the spacer height and the spacer thickness means the same thing.

The term “area” of an object in a CROF process refers to, unless specifically stated, the area of the object that is parallel to a surface of the plate. For example, spacer area is the area of the spacer that is parallel to a surface of the plate.

The term “lateral” or “laterally” in a CROF process refers to, unless specifically stated, the direction that is parallel to a surface of the plate.

The term “width” of a spacer in a CROF process refers to, unless specifically stated, a lateral dimension of the spacer.

The term “a spacer inside a sample” means that the spacer is surrounded by the sample (e.g. a pillar spacer inside a sample).

The term “critical bending span” of a plate in a CROF process refers the span (i.e. distance) of the plate between two supports, at which the bending of the plate, for a given flexible plate, sample, and compression force, is equal to an allowed bending. For example, if an allowed bending is 50 nm and the critical bending span is 40 um for a given flexible plate, sample, and compression force, the bending of the plate between two neighboring spacers 40 um apart will be 50 nm, and the bending will be less than 50 nm if the two neighboring spacers is less than 40 um.

The term “flowable” for a sample means that when the thickness of the sample is reduced, the lateral dimension increases. For an example, a stool sample is regarded flowable.

In some embodiments of the present invention, a sample under a CROF process do not to be flowable to benefit from the process, as long as the sample thickness can be reduced under a CROF process. For an example, to stain a tissue by put a dye on a surface of the CROF plate, a CROF process can reduce the tissue thickness and hence speed up the saturation incubation time for staining by the dye.

The terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers; and the terms refer to a device that comprises a first plate and a second plate that are movable relative to each other into different configurations (including an open configuration and a closed configuration), and that comprises spacers (except some embodiments of the COF) that regulate the spacing between the plates. The term “X-plate” refers to one of the two plates in a CROF card, wherein the spacers are fixed to this plate. More descriptions of the COF Card, CROF Card, and X-plate are described in the provisional application Ser. Nos. 62/456,065, filed on Feb. 7, 2017, which is incorporated herein in its entirety for all purposes.

### (1) Dimensions

The devices, apparatus, systems, and methods herein disclosed can include or use a QMAX device, which can comprise plates and spacers. In some embodiments, the dimension of the individual components of the QMAX device and its adaptor are listed, described and/or summarized in PCT Application (designating U.S.) No. PCT/US2016/045437 filed on Aug. 10, 2016, and U.S. Provisional Application Nos. 62,431,639 filed on Dec. 9, 2016 and 62/456,287 filed on Feb. 8, 2017, which are all hereby incorporated by reference by their entireties.

In some embodiments, the dimensions are listed in the Tables below:

### Plates:

| Parameters | Embodiments | Preferred Embodiments |
|---|---|---|
| Shape | round, ellipse, rectangle, triangle, polygonal, ring- | at least one of the two (or |
| | shaped, or any superposition of these shapes; the | more) plates of the QMAX |
| | two (or more) plates of the QMAX card can have | card has round corners for |
| | the same size and/or shape, or different size and/ | user safety concerns, wherein |
| | or shape; | the round corners have a |
| | | diameter of 100 um or less, 200 |
| | | um or less, 500 um or less, |
| | | 1 mm or less, 2 mm or less, 5 |
| | | mm or less, 10 mm or less, 50 mm |
| | | or less, or in a range between |
| | | any two of the values. |
| Thickness | the average thickness for at least one of the plates | For at least one of the |
| | is 2 nm or less, 10 nm or less, 100 nm or less, 200 | plates is in the range of 0.5 |
| | nm or less, 500 nm or less, 1000 nm or less, 2 μm | to 1.5 mm; around 1 mm; in |
| | (micron) or less, 5 μm or less, 10 μm or less, 20 | the range of 0.15 to 0.2 mm; |
| | μm or less, 50 μm or less, 100 μm or less, 150 μm | or around 0.175 mm |
| | or less, 200 μm or less, 300 μm or less, 500 μm or | |
| | less, 800 μm or less, 1 mm (millimeter) or less, 2 | |
| | mm or less, 3 mm or less, 5 mm or less, 10 mm or | |
| | less, 20 mm or less, 50 mm or less, 100 mm or less, | |
| | 500 mm or less, or in a range between any two of | |
| | these values | |
| Lateral Area | For at least one of the plate is 1 mm2 (square | For at least one plate of the |
| | millimeter) or less, 10 mm2 or less, 25 mm2 or | QMAX card is in the range of |
| | less, 50 mm2 or less, 75 mm2 or less, 1 cm2 | 500 to 1000 mm²; or around |
| | (square centimeter) or less, 2 cm2 or less, 3 cm2 | 750 mm². |
| | or less, 4 cm2 or less, 5 cm2 or less, 10 cm2 or | |
| | less, 100 cm2 or less, 500 cm2 or less, 1000 cm2 | |
| | or less, 5000 cm2 or less, 10,000 cm2 or less, | |
| | 10,000 cm2 or less, or in a range between any two | |
| | of these values | |
| Lateral Linear | For at least one of the plates of the QMAX card is | For at least one plate of the |
| Dimension (width, | 1 mm or less, 5 mm or less, 10 mm or less, 15 mm | QMAX card is in the range of |
| length, or | or less, 20 mm or less, 25 mm or less, 30 mm or | 20 to 30 mm; or around 24 mm |
| diameter, etc.) | less, 35 mm or less, 40 mm or less, 45 mm or less, | |
| | 50 mm or less, 100 mm or less, 200 mm or less, | |
| | 500 mm or less, 1000 mm or less, 5000 mm or less, | |
| | or in a range between any two of these values | |
| Recess width | 1 um or less, 10 um or less, 20 um or less, 30 um | In the range of 1 mm to 10 |
| | or less, 40 um or less, 50 um or less, 100 um or | mm; Or About 5 mm |
| | less, 200 um or less, 300 um or less, 400 um or | |
| | less, 500 um or less, 7500 um or less, 1 mm or | |
| | less, 5 mm or less, 10 mm or less, 100 mm or less, | |
| | or 1000 mm or less, or in a range between any two | |
| | of these values. | |

### Hinge:

| | | Preferred |
|---|---|---|
| Parameters | Embodiments | Embodiments |
| Number | 1, 2, 3, 4, 5, or more | 1 or 2 |
| Length of | 1 mm or less, 2 mm or less, 3 mm or | In the range of |
| Hinge Joint | less, 4 mm or less, 5 mm or less, | 5 mm to 30 mm. |
| | 10 mm or less, 15 mm or less, 20 mm | |
| | or less, 25 mm or less, 30 mm or | |
| | less, 40 mm or less, 50 mm or less, | |
| | 100 mm or less, 200 mm or less, or | |
| | 500 mm or less, or in a range | |
| | between any two of these values | |
| Ratio (hinge | 1.5 or less, 1 or less, 0.9 or less, | In the range of |
| joint length | 0.8 or less, 0.7 or less, 0.6 or | 0.2 to 1; or |
| vs. aligning | less, 0.5 or less, 0.4 or less, 0.3 | about 1 |
| plate edge | or less, 0.2 or less, 0.1 or less, | |
| length | 0.05 or less or in a range between | |
| | any two of these values. | |
| Area | 1 mm² or less, 5 mm² or less, 10 | In the range of |
| | mm² or less, 20 mm² or less, 30 mm² | 20 to 200 mm²; or |
| | or less, 40 mm² or less, 50 mm² or | about 120 mm² |
| | less, 100 mm² or less, 200 mm² or | |
| | less, 500 mm² or less, or in a | |
| | range between any of the two values | |
| Ratio (hinge | 1 or less, 0.9 or less, 0.8 or less, | In the range of |
| area vs. | 0.7 or less, 0.6 or less, 0.5 or | 0.05 to 0.2, |
| plate area) | less, 0.4 or less, 0.3 or less, 0.2 | around 0.15 |
| | or less, 0.1 or less, 0.05 or less, | |
| | 0.01 or less or in a range between | |
| | any two of these values | |
| Max. Open | 15 or less, 30 or less, 45 or less, | In the range of |
| Degree | 60 or less, 75 or less, 90 or less, | 90 to 180 degrees |
| | 105 or less, 120 or less, 135 or | |
| | less, 150 or less, 165 or less, 180 | |
| | or less, 195 or less, 210 or less, | |
| | 225 or less, 240 or less, 255 or | |
| | less, 270 or less, 285 or less, 300 | |
| | or less, 315 or less, 330 or less, | |
| | 345 or less or 360 or less degrees, | |
| | or in a range between any two of | |
| | these values | |
| No. of | 1, 2, 3, 4, 5, or more | 1 or 2 |
| Layers | | |
| Layer | 0.1 um or less, 1 um or less, 2 um | In the range of |
| thickness | or less, 3 um or less, 5 um or less, | 20 um to 1 mm; |
| | 10 um or less, 20 um or less, 30 um | or Around 50 um |
| | or less, 50 um or less, 100 um or | |
| | less, 200 um or less, 300 um or less, | |
| | 500 um or less, 1 mm or less, 2 mm | |
| | or less, and a range between any | |
| | two of these values | |
| Angle- | Limiting the angle adjustment with | No more than ±2 |
| maintain- | no more than ±90, ±45, ±30, ±25, | |
| ing | ±20, ±15, ±10, ±8, ±6, ±5, ±4, ±3, | |
| | ±2, or ±1, or in a range between | |
| | any two of these values | |

### Notch:

| | | Preferred |
|---|---|---|
| Parameters | Embodiments | Embodiments |
| Number | 1, 2, 3, 4, 5, or more | 1 or 2 |
| Shape | round, ellipse, rectangle, triangle, | Part of a circle |
| | polygon, ring-shaped, or any | |
| | superposition or portion of these | |
| | shapes. | |
| Positioning | Any location along any edge except | |
| | the hinge edge, or any corner joint | |
| | by non-hinge edges | |
| Lateral | 1 mm or less, 2.5 mm or less, 5 mm | In the range of 5 |
| Linear | or less, 10 mm or less, 15 mm or | mm to 15 mm; or |
| Dimension | less, 20 mm or less, 25 mm or less, | about 10 mm |
| (Length | 30 mm or less, 40 mm or less, 50 mm | |
| along the | or less, or in a range between any | |
| edge, radius, | two of these values | |
| etc.) | | |
| Area | 1 mm² (square millimeter) or less, | In the range of 10 |
| | 10 mm² or less, 25 mm² or less, | to 150 mm²; or |
| | 50 mm² or less, 75 mm² or less | about 50 mm² |
| | or in a range between any two of these | |
| | values. | |

### Trench:

| | | Preferred |
|---|---|---|
| Parameters | Embodiments | Embodiments |
| Number | 1, 2, 3, 4, 5, or more | 1 or 2 |
| Shape | Closed (round, ellipse, rectangle, | |
| | triangle, polygon, ring-shaped, or | |
| | any superposition or portion of | |
| | these shapes) or open-ended | |
| | (straight line, curved line, arc, | |
| | branched tree, or any other shape | |
| | with open endings); | |
| Length | 0.001 mm or less, 0.005 mm or less, | |
| | 0.01 mm or less, 0.05 mm or less, | |
| | 0.1 mm or less, 0.5 mm or less, 1 | |
| | mm or less, 2 mm or less, 5 mm or | |
| | less, 10 mm or less, 20 mm or less, | |
| | 50 mm or less, 100 mm or less, or | |
| | in a range between any two of these | |
| | values | |
| Cross- | 0.001 mm² or less, 0.005 mm² or less, | |
| sectional | 0.01 mm² or less, 0.05 mm² or less, | |
| Area | 0.1 mm² or less, 0.5 mm² or less, 1 | |
| | mm² or less, 2 mm² or less, 5 mm² or | |
| | less, 10 mm² or less, 20 mm² or less, | |
| | or in a range between any two of | |
| | these values. | |
| Volume | 0.1 uL or more, 0.5 uL or more, 1 uL | In the range of 1 |
| | or more, 2 uL or more, 5 uL or more, | uL to 20 uL; or |
| | 10 uL or more, 30 uL or more, 50 uL | About 5 uL |
| | or more, 100 uL or more, 500 uL or | |
| | more, 1 mL or more, or in a range | |
| | between any two of these values | |

### Receptacle Slot

| | | Preferred |
|---|---|---|
| Parameters | Embodiments | Embodiments |
| Shape of | round, ellipse, rectangle, triangle, | |
| receiving area | polygon, ring-shaped, or any | |
| | superposition of these shapes; | |
| Difference | 100 nm, 500 nm, 1 um, 2 um, 5 um, 10 | In the range |
| between sliding | um, 50 um, 100 um, 300 um, 500 um, 1 | of 50 to 300 |
| track gap size | mm, 2 mm, 5 mm, 1 cm, or in a range | um; or |
| and card | between any two of the values. | about 75 um |
| thickness | | |
| Difference | 1 mm² (square millimeter) or less, 10 | |
| between | mm² or less, 25 mm² or less, 50 mm² or | |
| receiving | less, 75 mm² or less, 1 cm² (square | |
| area and card | centimeter) or less, 2 cm² or less, 3 | |
| area | cm² or less, 4 cm² or less, 5 cm² or | |
| | less, 10 cm² or less, 100 cm² or less, | |
| | or in a range between any of the two | |
| | values. | |

### (2) Cloud

The devices/apparatus, systems, and methods herein disclosed can employ cloud technology for data transfer, storage, and/or analysis. The related cloud technologies are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the cloud storage and computing technologies can involve a cloud database. Merely by way of example, the cloud platform can include a private cloud, a public cloud, a hybrid cloud, a community cloud, a distributed cloud, an inter-cloud, a multi-cloud, or the like, or any combination thereof. In some embodiments, the mobile device (e.g. smartphone) can be connected to the cloud through any type of network, including a local area network (LAN) or a wide area network (WAN).

In some embodiments, the data (e.g. images of the sample) related to the sample is sent to the cloud without processing by the mobile device and further analysis can be conducted remotely. In some embodiments, the data related to the sample is processed by the mobile device and the results are sent to the cloud. In some embodiments, both the raw data and the results are transmitted to the cloud.

What is claimed is:

1. An apparatus for counting cells in a sample, comprising:
(a) a sample holder, comprising a first plate, a second plate, and spacers, a first plate, a second plate, and spacers, wherein:
i. the plates are movable relative to each other into different configurations;
ii. one or both plates are flexible;
iii. each of the plates has, on its respective surface, a sample contact area for contacting a sample that contains or is suspected containing cells to be counted,
iv. one or both plates are transparent;
v. one or both of the plates comprise the spacers that are fixed with a respective sample contact area, wherein the spacers have predetermined inter-spacer distance and a predetermined substantially uniform height, wherein at least one of the spacers is inside the sample contact area, and wherein the Young's modulus of the spacers times the filling factor of the spacers is equal or larger than 2 MPa;
wherein the filling factor is the ratio of the spacer contact area to the total plate area;
wherein one of the configurations is an open configuration, in which: the two plates are separated apart, the spacing between the plates is not regulated by the spacers, and the sample is deposited on one or both of the plates; and
wherein another of the configurations is a closed configuration which is configured after the sample deposition in the open configuration; and in the closed configuration: at least part of the sample is compressed by the two plates into a thin layer of uniform thickness of 200 um or less, wherein the uniform thickness of the layer is confined by the sample contact surfaces of the plates and is regulated by the plates and the spacers;
(b) an imager that is configured to an area (AOI—area of interest) of the sample; and
(c) a computer readable storage medium or memory storage unit comprising a computer program, wherein the computer program comprises an algorithm using a machine learning model for detecting and counting the cells from the image, and wherein the machine learning model has been trained using the images of the sample contact area, where the image include an image of the spacers, and wherein the spacer is configured as a scale marker, image marker, or a location marker.

2. A method for counting cells in a sample, comprising:
(a) receiving a sample that contains or suspected containing the cells to be detected and counted;
(b) loading the sample into a sample holder to make the sample into a thin layer;
(c) imaging, using an imager, an area (AOI—area of interest) of the sample in the sample holder;
(d) analyzing the images in (c) to detect and/or counting the cells; wherein a computer readable storage medium or memory storage unit comprising a computer program, wherein the computer program comprises an algorithm using a machine learning model for detecting and counting the cells from the image, and wherein the machine learning model has been trained using the images of the sample contact area, where the image include an image of the spacers, and wherein the spacer is configured as a scale marker, image marker, or a location marker;
wherein the sample holder, comprising a first plate, a second plate, and spacers,
wherein:
i. the plates are movable relative to each other into different configurations;
ii. one or both plates are flexible;
iii. each of the plates has, on its respective surface, a sample contact area for contacting a sample that contains or is suspected containing cells to be counted,
iv. one or both plates are transparent;
v. one or both of the plates comprise the spacers that are fixed with a respective sample contact area, wherein the spacers have a pillar shape, a substantially flat top surface, a predetermined substantially uniform height, wherein at least one of the spacers is inside the sample contact area, and wherein the Young's modulus of the spacers times the filling factor of the spacers is equal or larger than 2 MPa;
wherein the filling factor is the ratio of the spacer contact area to the total plate area;
wherein one of the configurations is an open configuration, in which: the two plates are separated apart, the spacing between the plates is not regulated by the spacers, and the sample is deposited on one or both of the plates; and
wherein another of the configurations is a closed configuration which is configured after the sample deposition in the open configuration; and in the closed configuration: at least part of the sample is compressed by the two plates into a thin layer of uniform thickness of 200 um or less, wherein the uniform thickness of the layer is confined by the sample contact surfaces of the plates and is regulated by the plates and the spacers.

3. The apparatus and method of any prior claim, wherein the cells are blood cells.

4. The apparatus and method of any prior claim, wherein the imaging is capable of imaging the local areas of the sample area.

5. The apparatus and method of any prior claim, wherein it further comprising a step of analyzing cell location, count, concentration of the detected cells.

6. The method of any prior claim, further comprising using the prediction to generate a heatmap.

7. The method of any prior claim, further comprising the step of storing the center location, count, and concentration of the blood cells in a storage device.

8. The method of any prior claim, further comprising the step of displaying the test results on the screen of a computer or a mobile device.

9. The method of any prior claim, wherein the annotating step comprising:
(a) collecting a plurality of the pseudo-2D images over multiple AoIs; and
(b) labeling the blood cell in the image to generate the annotated data set.

10. The method of any prior claim, wherein the pseudo-2D data is used to detect local signal peaks with:
(a) a signal list processing, or
(b) a local searching processing; and
(c) calculating the amount of blood cells being captured based on local signal peak information and the sample volume associated with the AoI in the assay.

11. The method of any prior claim, wherein the signal list processing comprises:
(a) establishing a signal list by detecting local peaks from the 2-D data array;
(b) calculating a local area surrounding the detected local peak; and
(c) removing the detected peak and the local area data into the signal list in rank order; and
(d) sequentially and repetitively removing highest signals from the signal list and signals from around the highest signal, thus detecting local signal peaks.

12. The method of any prior claim, wherein the local search process comprises:
(a) searching for a local maximal value in the 2-D data array by starting from a random point;
(b) calculating the local area surrounding the peak but with smaller value;
(c) removing the local maximal value and the surrounding smaller values from the 2-D data array; and
(d) repeating steps a-c to detect local signal peaks.

13. A system, comprising a QMAX device; an imager; and a computing unit, wherein:
(a) the QMAX device is configured to compress at least part of a test sample into a layer of highly uniform thickness;
(b) the imager is configured to produce an image of the sample at the layer of uniform thickness, wherein the image includes detectable signals from analytes in the test sample;
(c) the computing unit is configured to:
i. receive the image from the imager;
ii. analyze the image with a detection model and generate a 2-D data array of the image, wherein the 2-D data array includes the probability or likelihood data of the analyte for being at each location in the image, and the detection model is established through a training process that comprises:
a. feeding an annotated data set to a convolutional neural network, wherein the annotated data set is from samples that are the same type as the test sample and for the same analyte; and
b. training and establishing the detection model by convolution;
c. in testing, feeding the data to the model, generating and analyzing the 2-D data array to detect local signal peaks with signal list processing, or local search processing to detect the analyte; and
d. calculating the amount of the analyte being detected based on local signal peak information and the analyte relation to the assay volume.

14. The method and system of any prior claim, wherein the imager comprises a camera.

15. The method and system of any prior claim, wherein the camera is part of a mobile communication device such as a smart phone.

16. The method and system of prior claim, wherein the computing unit is part of a mobile communication device.

17. The method and system of any prior claim, wherein a method of mixture of computer vision and deep learning for data analysis is used, comprising:
(a) receiving an image of a test sample, wherein the sample is loaded into a QMAX device and the image is taken by an imager connected to the QMAX device, wherein the image includes detectable signals from an analyte in the test sample;
(b) analyzing the image with a detection algorithm that finds possible candidate based on the characteristics of analytes;
(c) analyzing the image with a localization algorithm that locates each possible candidate of analytes by providing its boundary or a tight bounding box containing it;
(d) analyzing the image with a deep learning algorithm that classifies each possible candidate as a true analyte and false analyte;
(e) outputting the locations of true analytes, the total count of true analytes and the concentration of the analytes in the assay.

18. The method and system of any prior claim, where the detection is based on the analyte structure (such as edge detection, line detection, circle detection, etc.).

19. The method and system of any prior claim, where the detection is based on the connectivity (such as blob detection, connect components, contour detection, etc.).

20. The method and system of any prior claim, where the connectivity is blob detection, connect components, or contour detection.

21. The method and system of any prior claim, where the detection is based on intensity, color, shape using schemes such as adaptive thresholding.

22. The method and system of any prior claim, where the detection is enhanced by a pre-processing scheme.

23. The method and system of any prior claim, where the localization is based on an object segmentation algorithm selected from the group consisting of adaptive thresholding, background subtraction, flood fill, mean shift, and watershed.

24. The method and system of any prior claim, the localization is combined with detection to produce the detection results along with the location of each possible candidates of analytes.

25. The method and system of any prior claim, where the detection and classification are based on machine learning.

26. The method and system of claim 2, wherein the machine learning is a convolutional neural network.

27. The method and system of any prior claim, wherein one plate of the said device is transparent, so that the AoI (area-of-interest) on the said plate can be imaged to reveal the pseudo-2D layer of the analytes sandwiched between the two narrowly spaced plates.

28. The method and system of any prior claim, wherein the is diagnostic, chemical or biological test generally.

29. A machine learning framework at microscopic cell distribution level to detect, locate, count and obtain all types of analyte concentrations with method of deep learning for data analysis, comprising:
(a) receiving an image of a test sample, wherein the sample is loaded into a QMAX device and the image is taken by an imager connected to the QMAX device, wherein the image includes detectable signals from an analyte in the test sample;
(b) analyzing the image with a detection model and generating a 2-D data array of the image, wherein the 2-D data array includes probability data of the analyte for each location in the image, and the detection model is established through a training process that comprises:
i. feeding an annotated data set to a convolutional neural network, wherein the annotated data set is from samples that are the same type as the test sample and containing the same type of analytes for assaying; and
ii. training and establishing the detection model with convolution; and
(c) analyzing the 2-D data array to detect local signal peaks with:
i. signal list process, or
ii. local searching process; and
(d) calculating the amount of the analytes based on local signal peak information.

30. The machine learning framework of claim 29, wherein the signal list process comprises:
(a) establishing a signal list by iteratively detecting local peaks from the 2-D data array, calculating a local area surrounding the detected local peak, and removing the detected peak and the local area data into the signal list in order; and
(b) sequentially and repetitively removing highest signals from the signal list and signals from around the highest signal, thus detecting local signal peaks.

31. The machine learning framework of claim 29, wherein the local search process comprises:
(a) searching for a local maximal value in the 2-D data array by starting from a random point;
(b) calculating the local area surrounding the peak but with smaller value;
(c) removing the local maximal value and the surrounding smaller values from the 2-D data array; and
(d) repeating steps i-iii to detect local signal peaks.

32. The machine learning framework of claim 29, wherein the annotated data set is partitioned before annotation.

33. A system for data analysis, comprising:
a QMAX device; an imager; and a computing unit, wherein:
(a) the QMAX device is configured to compress at least part of a test sample into a layer of highly uniform thickness;
(b) the imager is configured to produce an image of the sample at the layer of uniform thickness, wherein the image includes detectable signals from an analyte in the test sample;
(c) the computing unit is configured to:
i. receive the image from the imager;
ii. analyze the image with a detection model and generate a 2-D data array of the image, wherein the 2-D data array includes probability data of the analyte for each location in the image, and the detection model is established through a training process that comprises: feeding an annotated data set to a convolutional neural network, wherein the annotated data set is from samples that are the same type as the test sample and contain the same type of analytes for assaying; training and establishing the detection model with convolution; and
iii. analyzing the 2-D data array to detect local signal peaks with signal list process, or local searching process; and
iv. calculate the amount of the analytes based on local signal peak information.

34. The system of claim 33, wherein the imager comprises a camera.

35. The system of claim 33, wherein the camera is part of a mobile communication device.

36. The system of claim 33, wherein the computing unit is part of a mobile communication device.

37. A method of mixture of computer vision and deep learning for data analysis, comprising:
(a) receiving an image of a test sample, wherein the sample is loaded into a QMAX device and the image is taken by an imager connected to the QMAX device, wherein the image includes detectable signals from an analyte in the test sample;
(b) analyzing the image with a detection algorithm that finds possible candidate based on the characteristics of analytes;
(c) analyzing the image with a localization algorithm that locates each possible candidate of analytes by providing its boundary or a tight bounding box containing it;
(d) analyzing the image with a deep learning algorithm that classifies each possible candidate as a true analyte and false analyte; and
(e) outputting the locations of true analytes and the total count of true analytes.

38. The system of claim 37, where the detection is based on the analyte structure (such as edge detection, line detection, circle detection, etc.).

39. The system of claim 37, where the detection is based on the connectivity (such as blob detection, connect components, contour detection, etc.).

40. The system of claim 37, where the detection is based on intensity, color, shape using schemes such as adaptive thresholding, etc.

41. The system of claim 37, where the detection is enhanced by a pre-processing scheme.

42. The system of claim 37, where the localization is based on object segmentation algorithms, such as adaptive thresholding, background subtraction, floodfill, mean shift, watershed, etc.

43. The system of claim 37, where the localization is combined with detection to produce the detection results along with the location of each possible candidates of analytes.

44. The system of claim 37, where the classification is based on deep learning, such as a convolutional neural network.

45. A non-transitory computer readable medium embodying a program of instructions executable by machine to perform steps for supporting a workflow, the steps comprising:
(a) receiving an image containing a pseudo-2D object of an analyte;
(b) generating a list of the pseudo-2D object from the image;
(c) annotating a data set of the analyte based on:
(i) concentration of the analyte, and
(ii) location of the analyte;
(d) feeding said annotated image into a convolutional neural network to analyze the pseudo-2D data; and
(e) performing machine learning to generate a detection model useful for making pixel-level prediction on said image.

46. The non-transitory computer readable medium of claim 45, further comprising the step of using the prediction to generate a heatmap.

47. The non-transitory computer readable medium of claim 45, further comprising the step of storing the center location, count, and concentration of the blood cells in a storage device.

48. The non-transitory computer readable medium of claim 45, further comprising the step of displaying the test results on the screen of a computer or a mobile device.
